(19) United States
(12) Patent Application Publication
(10) Pub. No.: US 2019/0160149 A1
(43) Pub. Date: May 30, 2019

(54) PHARMACEUTICAL COMPOSITION FOR PREVENTING OR TREATING CARTILAGE DISEASES

(71) Applicant: UNIST (ULSAN NATIONAL INSTITUTE OF SCIENCE AND TECHNOLOGY), Ulsan (KR)

(72) Inventors: Tae Joo Park, Ulsan (KR); Eun Kyung Song, Ulsan (KR)

(21) Appl. No.: 16/185,105

(22) Filed: Nov. 9, 2018

(30) Foreign Application Priority Data
Nov. 30, 2017 (KR) 10-2017-0163527
Aug. 30, 2018 (KR) 10-2018-0102801

(51) Int. Cl. A61K 38/17; A61P 19/02; A61K 35/32; A61K 35/761

(52) U.S. Cl. CPC A61K 38/1777; A61K 35/761; A61K 35/32; A61P 19/02

(57) ABSTRACT

A pharmaceutical composition for preventing or treating cartilage diseases, and pharmaceutical preparation that includes the pharmaceutical composition as an active ingredient are provided. The pharmaceutical composition includes, as an active ingredient, at least one of an integrin beta-like 1 (ITGBL1) protein, ITGBL1 DNA or RNA encoding the

### CROSS-REFERENCE TO RELATED APPLICATION(S)

This application claims the priority benefit of Korean Patent Application No. 10-2017-0163527 filed on Nov. 30, 2017, and Korean Patent Application No. 10-2018-0102801 filed on Aug. 30, 2018, in the Korean Intellectual Property Office, the disclosures of which are incorporated herein by reference for all purposes.

### BACKGROUND

### 1. Field of the Invention

One or more example embodiments relate to a pharmaceutical composition for preventing or treating cartilage diseases that includes at least one of an integrin beta-like 1 (ITGBL1) protein and ITGBL1 encoding the ITGBL1 protein as an active ingredient, and to a pharmaceutical preparation that includes the pharmaceutical composition as an active ingredient.

### 2. Description of the Related Art

Degenerative cartilage diseases or osteoarthritis are characterized by pain or stiffness in the joint caused by gradual cartilage loss after injury or by aging process. For treatment of patients with an early-stage cartilage disease, analgesic anti-inflammatory drugs to suppress an inflammation and pain are mainly used. For treatment of patients with a middle-stage cartilage disease, analgesic anti-inflammatory drugs and hyaluronic acid are periodically injected, and gene medicines or cell therapeutic agents for promoting regeneration of cartilage are used. For treatment of patients with an end-stage cartilage disease, artificial joint surgeries are mainly used.

TGF-β is known as a gene medicine used in combination with cell therapeutic agents that promote cartilage regeneration. Invossa™ recently released from Kolon Life Science is known to contain TGF-β that is a gene medicine.

Current treatments suggested for cartilage regeneration include implantation of a stem cell therapeutic agent or an autologous cell therapeutic agent. However, the above two therapeutic agents have failed to successfully enter the market to date due to insufficient cartilage regeneration and high procedure costs.

Therefore, to fundamentally treat cartilage diseases, an additional cartilage damage caused by an inflammation needs to be inhibited, and a treatment to regenerate damaged cartilage also needs to be performed. However, most current medications only relieve symptoms or attenuate cartilage loss, and no treatments can effectively regenerate damaged cartilage.

### SUMMARY

Example embodiments provide a pharmaceutical composition for preventing or treating cartilage diseases that includes, as an active ingredient, at least one of an integrin beta-like 1 (ITGBL1) protein, ITGBL1 DNA or RNA encoding the ITGBL1 protein, a recombinant vector including the ITGBL1, and recombinant cells transformed with the recombinant vector.

Example embodiments provide an inhibitor of integrin activation that includes, as an active ingredient, at least one of an ITGBL1 protein, ITGBL1 DNA or RNA encoding the ITGBL1 protein, a recombinant vector including the ITGBL1 DNA sequences, and recombinant cells transformed with the recombinant vector, and an in vitro reagent composition for inhibiting integrin activation.

Example embodiments provide a pharmaceutical preparation that includes the pharmaceutical composition as an active ingredient.

Example embodiments provide a method of preventing or treating cartilage diseases that includes administering a pharmaceutically effective amount of the pharmaceutical composition.

In an example embodiment, an ITGBL1 protein that is expressed and secreted around chondrocytes was identified during a differentiation of chondrocytes (Example 1, and FIGS. 1A through 1E).

In an example embodiment, it is confirmed that when ITGBL1 is ectopically expressed into chondrocytes to increase expression of an ITGBL1 protein, a cartilage tissue is increased, and thus it is found that the ITGBL1 protein has an effect of promoting chondrogenesis (Examples 2 and 3, and FIGS. 2A through 2E and 3A through 3H).

In an example embodiment of the present disclosure, it is confirmed that when ITGBL1 is transfected into arthritis-induced chondrocytes to increase expression of an ITGBL1 protein, the ITGBL1 protein has an effect of promoting chondrogenesis (Example 4, and FIGS. 4A through 4D).

In an example embodiment, it is confirmed that when ITGBL1 is transfected into chondrocytes to increase expression of an ITGBL1 protein, the ITGBL1 protein has an effect of reducing expression of an inflammatory factor (for example, MMP3, MMP13, and the like) in arthritis-induced chondrocytes inflammation (Example 5, and FIGS. 4D and 4E) and an effect of suppressing osteoarthritis in mouse osteoarthritis model (Example 5, and FIGS. 4G and 4H).

In an example embodiment, it is confirmed that when expression of an ITGBL1 protein is inhibited by injecting an shRNA containing an adenoviral vector into a knee joints using a mouse model, a degenerative joint disease is induced (Example 8, and FIGS. 9D and 9E).

In an example embodiment, it is confirmed that when expression of an ITGBL1 protein is increase by injecting an adenoviral vector containing ITGBL1 into a knee joint of a mouse model on which a meniscectomy was performed, cartilage loss of the knee joints is attenuated (Example 4, and FIGS. 4G and 4H).

In an example embodiment, it is confirmed that an ITGBL1 protein inhibits activation of integrin-beta 1 (Example 6, and FIGS. 5A through 5G and 6A through 6F). In an example embodiment, it is confirmed that ITGBL1 promotes expression of a chondrogenic gene (for example, Sox9 and Col2a1) via integrin inactivation (Example 7, and FIGS. 7A through 7G).

In an example embodiment, it is confirmed that ITGBL1 reduces expression of MMP3 and MMP13 which is increased by a treatment with fragmented fibronectin (29-kDa Fn-fs) that are known to cause cartilage degeneration in chondrocytes. Furthermore, when integrins are activated through a treatment with Mn²⁺ or DTT after ITGBL1 transfection, the expression of MMP3 and MMP13 increases (Example 8, and FIGS. 8A through 8E).

In an example embodiment, it is confirmed that arthritis-like cartilage damage was developed when expression of ITGBL1 was inhibited in a knee joint of a mouse, and that arthritis damage was partially recovered when ATN-161, an integrin-beta 1 inhibitor was injected into the knee joint cavity in which the expression of ITGBL1 was inhibited (Example 8, and FIGS. 9D and 9E)

Thus, the ITGBL1 protein have an effect of promoting chondrogenesis while inhibiting an inflammation of cartilage diseases, and thus a composition including the ITGBL1 protein may be used to prevent or treat cartilage diseases.

Hereinafter, example embodiments will be described in more detail.

According to an aspect, there is provided a pharmaceutical composition for preventing or treating cartilage diseases that includes, as an active ingredient, at least one of an ITGBL1 protein, ITGBL1 DNA or RNA encoding the ITGBL1 protein, a recombinant vector including the ITGBL1 DNA sequences, and recombinant cells transformed with the recombinant vector. According to a related art, the ITGBL1 is known to function to regulate a marker of a metastatic cancer, or WNT/PCP signaling.

In the present disclosure, an ITGBL1 protein has an effect of promoting chondrogenesis while inhibiting an inflammation of cartilage diseases. The effect was verified as a phenomenon occurring due to a function of ITGBL1 inhibiting integrin activation. Thus, a composition including the ITGBL1 protein may have an effect of preventing or treating cartilage diseases, and ITGBL1 DNA or RNA and an ITGBL1 protein may be utilized as inhibitors for inhibiting integrin activation, and accordingly the composition may be used for diseases known to be caused by excessive activation of integrin. The ITGBL1 protein may have an amino acid sequence of SEQ ID NO: 25 and the ITGBL1 encoding the ITGBL1 protein may have a nucleotide sequence of SEQ ID NO: 26.

The term “protein” as used herein refers to a molecule including a polymer of amino acids linked together by a peptide bond(s). The protein may be a polypeptide including at least two amino acids.

The ITGBL1 protein may be derived from vertebrata, for example, mammals such as humans and mice, and amphibians such as frogs. The ITGBL1 protein may include, for example, but is not limited to, at least one of human ITGBL1 (for example, GenBank Accession Nos. NP_001258683.1 (coding mRNA (cDNA): NM_001271754.1), NP_001258684.1 (coding mRNA (cDNA): NM_01271755.1), NP_001258685.1 (coding mRNA (cDNA): NM_001271756.1), P_004782.1 (coding mRNA (cDNA): NM_004791.2), XP_005254157.1 (coding mRNA (cDNA): XM_005254100.4), and the like), mouse ITGBL1 (for example, GenBank Accession Nos. NP_663442.2 (coding mRNA (cDNA): NM_145467.2), XP_006518984.1 (coding mRNA (cDNA): XM_006518921.3), and the like), and frog ITGBL1 (for example, GenBank Accession No. NP_001084310.1 (coding mRNA (cDNA): NM_001090841.1), and the like).

The term “vector” refers to a means for expressing a gene of interest in a host cell. A vector may include elements required for expression of a gene of interest, and may include, for example, a replication origin, a promotor, an operator gene, a transcriptional terminator, and the like. In addition, the vector may further include an appropriate enzyme site (for example, a restriction enzyme site) for an introduction into a genome of a host cell, and/or a selection marker for confirming a successful introduction into a host cell, and/or a ribosome binding site (RBS) for translation to a protein, an internal ribosome entry site (IRES), and the like. A vector may be engineered using a typical genetic engineering method to include a fusion polynucleotide (for example, a fusion promoter) as a promoter. The vector may further include a transcription control sequence (for example, an enhancer, and the like), in addition to the promotor.

The recombinant vector may be a viral vector or a nonviral vector. The viral vector may include, for example, but is not limited to, an adenovirus vector, an adeno-associated virus vector, a helper-dependent adenovirus vector, and a retroviral vector.

The recombinant vector may be implemented using various methods known in the art.

According to another aspect, there is provided recombinant cells transformed with the recombinant vector.

The recombinant cells may be mammalian cells. The mammalian cells may be selected from, but are not limited to, human cells (for example, adipose-derived stem cells, bone marrow-derived stem cells, placenta-derived stem cells, other induced pluripotent stem cells, chondrocytes, fibroblasts, and the like).

The recombinant vector may be transferred (or introduced) into a cell using a transfer method that is well known in the art. The transfer method may include, for example, a microinjection, a calcium phosphate precipitation, an electroporation, a liposome-mediated transfection and a gene bombardment. For example, the liposome-mediated transfection (using a lipofector reagent) may be used, but is not limited thereto.

The cartilage diseases may include, for example, degenerative arthritis, posttraumatic arthritis, or osteochondritis dissecans, but are not limited thereto.

In the present disclosure, ITGBL1 may be used in a form of DNA, RNA, or a protein, and may include genes derived from fish, amphibians, birds, and mammals. Also, ITGBL1 may include a nucleotide sequence of a wild-type ITGBL1 and an amino acid sequence variant.

A variant of an ITGBL1 protein or a variant of an amino acid sequence refers to a variation (for example, deletion and insertion) of an amino acid sequence of ITGBL1, and may include a change in chemical properties to change physiological activity and stability of a protein.

In the present disclosure, all schemes of artificially transferring ITGBL1 into cells may be used to increase expression of an ITGBL1 protein or ITGBL1.

In the present disclosure, at least one of an ITGBL1 protein, ITGBL1 DNA or RNA encoding the ITGBL1 protein, a recombinant vector including the ITGBL1 DNA sequences, and recombinant cells transformed with the recombinant vector may be used. In addition to at least one of the ITGBL1 protein, the ITGBL1 DNA or RNA encoding the ITGBL1 protein, the recombinant vector including the ITGBL1, and the recombinant cells transformed with the recombinant vector, an anti-inflammatory drug, hyaluronic acid, a cell therapeutic agent, and a stem cell therapeutic agent that are therapeutic agents for induction of cartilage regeneration and inhibition of an inflammation may be used together.

In a cell therapeutic agent of the present disclosure, cells used for cell therapy may refer to chondrocytes, and various cells associated with cartilage diseases, but example embodiments are not limited thereto.

In the stem cell therapeutic agent of the present disclosure, stem cells may include, for example, all types of stem cells having multipotency, for example, bone marrow-derived stem cells, adipose-derived stem cells, placenta-derived stem cells, induced pluripotent stem cells, and embryo-derived stem cells.

According to another aspect, there is provided a pharmaceutical preparation that includes the pharmaceutical composition as an active ingredient.

The pharmaceutical preparation may be formulated in a form of oral administration, such as, powders, granules, tablets, capsules, ointments, suspensions, emulsions, syrups, aerosols, and the like, or in a form of a parenteral administration, such as patches, suppositories and sterile injectable solutions.

The pharmaceutical preparation may further include an adjuvant, for example, a carrier, an excipient, a diluent, and the like, that are pharmaceutically suitable and physiologically acceptable. A carrier, an excipient and a diluent that may be included in the pharmaceutical composition may include, for example, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, and mineral oil. The pharmaceutical preparation may be formulated using typically used diluents or excipients, such as fillers, extenders, binders, wetting agents, disintegrating agents, surfactants, and the like.

For example, the pharmaceutical preparation may be provided for parenteral administration. The pharmaceutical preparation for parenteral administration may include, for example, a topical administration, such as liquids, gels, cleaning compositions, tablets for insertion, suppositories, creams, ointments, dressing solutions, spraying agents, or other coating agents, or a liquid-phase formulation, such as solutions, suspensions, or emulsions. The pharmaceutical preparation may include, for example, sterile aqueous solutions, nonaqueous solvents, suspensions, emulsions, freeze-dried preparations, suppositories, creams, ointments, jellies, foams, detergents, or inserts, and desirably, a skin external application, such as liquids, gels, cleaning compositions, tablets for insertion, and the like. The above formulation may be prepared by adding, to sterile water, a solubilizing agent, an emulsifier, a buffer for pH control, and the like. As a nonaqueous solvent and a suspension, propylene glycol, polyethylene glycol, vegetable oil, such as olive oil, injectable esters, such as ethyloleate, and the like, may be used.

Also, the pharmaceutical preparation may include a carrier that is added to the pharmaceutical composition to formulate the pharmaceutical composition. The carrier may include, for example, a binder, a lubricant agent, a suspension, a solubilizer, a buffer, a preservative, an antifriction, an isotonic agent, an excipient, a stabilizer, a dispersant, a suspending agent, a pigment, perfume, and the like.

In an example of applying the pharmaceutical preparation to a human, the pharmaceutical preparation may be administered alone, however, may be generally administered together with a selected pharmaceutical carrier based on a standard pharmaceutical practice and an administration route. For example, the pharmaceutical preparation may be orally, intrabuccally, or sublingually administered in a form of a tablet containing starch or lactose, or a capsule alone or containing an excipient, or an elixir containing flavoring or coloring chemicals, or suspension.

The description of the pharmaceutical composition for preventing or treating cartilage diseases may equally be applicable to the pharmaceutical preparation.

According to another aspect, there is provided an inhibitor of integrin activation that includes, as an active ingredient, at least one of an ITGBL1 protein, ITGBL1 DNA or RNA encoding the ITGBL1 protein, a recombinant vector including the ITGBL1 DNA sequences, and recombinant cells transformed with the recombinant vector.

The term “integrin” refers to a protein that connects a cell and an extracellular matrix (ECM), and includes two chains, that is, an a chain and a 13 chain. Based on a combination of the chains, at least “24” integrins may be present.

In particular, the inhibitor of integrin activation may be excellent in inhibition of activation of integrin beta. Thus, the inhibitor may be used to study mechanism of diseases caused by activation of integrin beta proteins, and to develop therapeutic agents, and the like. Examples of diseases induced by excessive activation of integrin may include, but are not limited to, cancer, irritable bowel syndrome, psoriasis, thrombosis, rheumatoid arthritis, osteoporosis.

According to another aspect, there is provided a method of preventing or treating cartilage diseases that includes administering a pharmaceutically effective amount of the pharmaceutical composition to a subject requiring prevention or treatment of cartilage diseases.

The subject may include, for example, but is not limited to, a subject (for example, animals other than humans) selected from vertebrata that include mammals such as humans, rodents such as mice, and amphibians such as frogs, and the like, or an organ, a tissue, a cell or a culture thereof isolated from the subject, and desirably, animals other than humans.

The cartilage diseases may include, for example, degenerative arthritis, posttraumatic arthritis, or osteochondritis dissecans, but are not limited thereto.

The description of the pharmaceutical composition for preventing or treating cartilage diseases may equally be applicable to the method of preventing or treating cartilage diseases.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1A illustrates an experiment to verify expression of integrin beta-like 1 (ITGBL1) in cartilage tissues during a Xenopus laevis embryogenesis, and FIGS. 1B and 1C illustrate results indicating whether ITGBL1 is expressed in chondrocytes of Xenopus laevis embryos according to an example embodiment. FIG. 1D illustrates results indicating that ITGBL1 is expressed in cartilage tissues based on a comparison between an expression pattern of Col2a1 that is a cartilage tissue marker gene and an expression pattern of ITGBL1, and FIG. 1E illustrates results indicating that ITGBL1 is strongly expressed at a cartilage tissue formation time (stage 30).

FIGS. 2A and 2C illustrate results indicating that cartilage tissues were reduced in size and cartilage tissue formation is significantly suppressed when expression of ITGBL1 in chondrocytes of Xenopus laevis embryos is inhibited, and that cartilage is formed normally and cartilage increases in size when the expression of ITGBL1 is increased, according to an example embodiment, and FIGS. 2B and 2D are graphs illustrating sizes of cartilages of FIGS. 2A and 2C, respectively. FIG. 2E illustrates results indicating that expression of Sox9 and Col2a1 that are markers of cartilage tissues is increased in embryos in which the expression of the ITGBL1 is increased.

FIGS. 3A and 3B illustrate results indicating that expression of ITGBL1 increases during a chondrocyte differentiation of each bone marrow-derived mesenchymal stem cell (BM-MSC) and that expression of ITGBL1 decreases during a bone tissue formation. FIGS. 3C and 3D illustrate results that a formation of a cartilage tissue is inhibited when expression of an ITGBL1 protein decreases in human chondrocytes. FIG. 3E illustrates results indicating that expression of Col2a1 and Sox9 that are genes promoting chondrogenesis gradually increases in response to a gradual increase in expression of ITGBL1 in a mouse chondrocyte line, and FIG. 3F illustrates results of a quantitative analysis of the expression of FIG. 3E using quantitative polymerase chain reaction (qPCR). FIG. 3G illustrates results indicating that a cartilage differentiation is promoted in response to an increase in expression of ITGBL1 of limb-bud mesenchyme that is a part of an arm and leg of a mouse embryo and differentiates into chondrocytes. FIG. 3H illustrates a result of a measurement of an amount of glycosaminoglycan (GAG) in the chondrocytes of FIG. 3G indicating that a cartilage formation is promoted by overexpression of ITGBL1.

FIGS. 4A through 4F illustrate results indicating that when expression of an ITGBL1 protein is increased in chondrocytes of a mouse in which an inflammation is caused by treatment with IL-1β, expression of Sox9 and Col2a1 that are chondrogenic factors is recovered and expression of MMP3 and MMP13 that are inflammatory factors is reduced, according to an example embodiment. FIGS. 4G and 4H illustrate results indicating that arthritis is not developed when expression of ITGBL1 is increased by injecting ITGBL1-containing adenovirus into knee joint cavities of mice with arthritis induced by damaging medial meniscus cartilage of knee joints of mice.

FIGS. 5A and 5C illustrate results indicating that an amount of focal adhesion complexes formed in a site in which integrin and a cell matrix bind significantly increases, by suppressing expression of ITGBL1 using ITGBL1-siRNA in a PC3 cell line, and FIG. 5B is a graph illustrating an intensity and the number of FAK puncta that is a marker of the focal adhesion complexes of FIG. 5A. FIG. 5D is a graph illustrating an intensity and the number of integrin-beta 1 puncta that is another marker of the focal adhesion complexes of FIG. 5C.

FIG. 5E illustrates results indicating that an amount of activated integrin-beta 1 increases when expression of ITGBL1 is inhibited and that the amount of activated integrin-beta 1 decreases when the expression of ITGBL1 is increased, and FIG. 5F illustrates results indicating, using fluorescent staining, that an amount of activated integrin-beta 1 increases when expression of ITGBL1 is reduced. FIG. 5G illustrates results indicating that integrin-beta 1 and ITGBL1 protein bind to each other, using a co-immunoprecipitation experiment.

FIG. 6A illustrates results indicating that when expression of ITGBL1 is increased (Itgbl1-O.E.), PC3 cells do not properly adhere onto a surface coated with fibronectin, but that when activation of integrins is increased by Mn²⁺ ions, the PC3 cells properly adhere onto the surface, and FIG. 6B illustrates a result of a measurement of a cell area of FIG. 6A. FIGS. 6C and 6D illustrate results indicating that when expression of ITGBL1 increases in BM-MSCs, a cell adhesion is inhibited. FIGS. 6E and 6F illustrate results of experiments of FIG. 6A performed in human chondrocytes, and the results of FIGS. 6E and 6F indicate that when expression of ITGBL1 is increased in human chondrocytes, a cell adhesion is inhibited and when activation of integrins is increased by Mn²⁺ ions, cells properly adhere onto the surface again.

FIG. 7A illustrates results indicating that expression of Sox9 and Col2a1 increases when expression of ITGBL1 increases in chondrocytes, but that the expression of Sox9 and Col2a1 decreases again when activation of integrins is increased through treatment with Mn²⁺ or DTT, and FIG. 7B illustrates a result of a quantitative analysis of the results of FIG. 7A using qPCR. FIG. 7C illustrates results indicating that a size of cartilage is increased by overexpression of ITGBL1 in a process of forming a cartilage tissue using an ATDC5 chondrocyte line. The size of cartilage is reduced by adding Mn²⁺ or DTT which activate integrins. FIGS. 7D and 7E illustrate analysis of the results of FIG. 7C based on a size of cartilage and an amount of GAG included in cartilage, respectively. FIG. 7F illustrates results indicating that expression of Sox9 increases when expression of integrin alpha and beta subunits of a table is inhibited. FIG. 7G illustrates results indicating that expression of Sox9 further increases when expression of integrin alpha and beta subunits is reduced in a state in which ITGBL1 is overexpressed.

FIGS. 8A through 8C illustrate results indicating that expression of MMP3 and MMP13 that promote decomposition of cartilage is increased in chondrocytes treated with 29-kDa fibronectin fragments (29-kDa Fn-fs) known to cause a cartilage degeneration of a patient with arthritis, but that the expression of MMP3 and MMP13 decreases again when expression of ITGBL1 is increased, and that the expression of MMP3 and MMP13 is increased again when integrins are activated through treatment with Mn²⁺ or DTT. FIGS. 8D and 8E illustrate results of a measurement of an adhesion level of 29-kDa Fn-fs to chondrocytes.

FIGS. 9A through 9C illustrate results indicating that expression of Mmp3 and Mmp13 increased by ITGBL1 depletion is not inhibited by treating subtype-specific integrin inhibitors Bio1211 (integrin-α4β1 inhibitor), obtustatin (integrin-α1β1 inhibitor), or ATN-161 (integrin-α5β1 inhibitor), but the expression of MMP3 and MMP13 is inhibited when a various types of integrin inhibitors are treated together, and that ATN-161 among integrin inhibitors is most effective. FIGS. 9D and 9E illustrate results indicating that articular cartilage deteriorates when expression of ITGBL1 is inhibited by injecting ITGBL1-shRN-containing adenovirus into knee joint cavities of mice, and that knee cartilage is recovered by injecting ATN-161 that is an integrin-beta 1 inhibitor into knee joint capsule in which the expression of ITGBL1 is inhibited.

FIG. 10 illustrates an example of a mechanism of promoting chondrogenesis and inhibiting an inflammation of cartilage diseases by inhibiting activation of integrins by expression of ITGBL1.

### DETAILED DESCRIPTION

Hereinafter, the present disclosure will be described in more detail with reference to examples. The following examples are given for the purpose of illustrating the present disclosure, and the scope of the present disclosure is not limited thereto.

### REFERENCE EXAMPLE 1

### Culture of Xenopus Laevis Embryos

Female Xenopus laevis were cultured in a 16° C. incubator (JSR, JSBI-150C), ovulation was induced by injecting 800 units of human chorionic gonadotropin (hCG, DS HCG Inj.) into the female Xenopus laevis. When the female Xenopus laevis began to lay eggs by hormones, the eggs were artificially squeezed and fertilized in vitro with male testes, followed by waiting for 30 minutes. Whether the eggs were fertilized was checked with naked eyes, the eggs were dejellied using 3% (w/v) cysteine (pH 7.8, Sigma:C7880), and Xenopus laevis embryos were cultured in 1/3× Marc's Modified Ringer's (MMR).

### REFERENCE EXAMPLE 2

### Cell Culture

Human PC3 cells, HEK293T cells, or human bone marrow-derived mesenchymal stem cells (hBMSCs) were cultured in 1% L-glutamine, 10% fetal bovine serum (FBS), 1% penicillin-streptomycin, an RPMI 1640 medium, a Dulbecco's modified Eagle medium (DMEM) and an α-MEM. To assess chondrogenesis of hBMSCs, pellet, micromass, or Transwell culture systems were employed.

Articular chondrocytes were isolated from femoral condyles and tibial plateaus of postnatal day 5 mice. Cartilage tissues were digested with 0.2% collagenase type II. Chondrocytes were maintained in a DMEM containing 10% FBS, penicillin and streptomycin.

Human chondrocytes were purchased from Cell Application, Inc. (San Diego, Calif., USA), and hBMSCs were purchased from the ATCC.

Mesenchymal cells obtained from embryos of ICR mice were digested with 1% trypsin and 0.2% collagenase type II and maintained to induce chondrogenesis and hypertrophic maturation. A total of 2×10⁷ cells/ml was suspended in a DMEM/F-12 medium (2:3) containing 10% (v/v) FBS. The cells were spotted as 20 pl drops on culture dishes and maintained for 6 days to induce chondrogenesis.

### EXAMPLE 1

### Confirmation of Expression of ITGBL1 in Chondrocytes

1-1. Confirmation of Expression Site of ITGBL1

To analyze genes expressed in pharyngeal arches of Xenopus laevis embryos, the following experiment was conducted.

The pharyngeal arches of Xenopus laevis embryos at stage 37 in which a face of an embryo is developed were dissected, and each of the dissected pharyngeal arches were divided into three parts in a cephalocaudal direction, and divided into two parts in a dorsoventral direction. RNA was extracted using a Trizol reagent (Sigma), an RNA-seq library was prepared from the extracted RNA using an Illumina TruSeq RNA Library Prep Kit, and a sequence analysis was commissioned by the Genome Sequencing Service Center (GSSC) at Stanford, U.S.A.

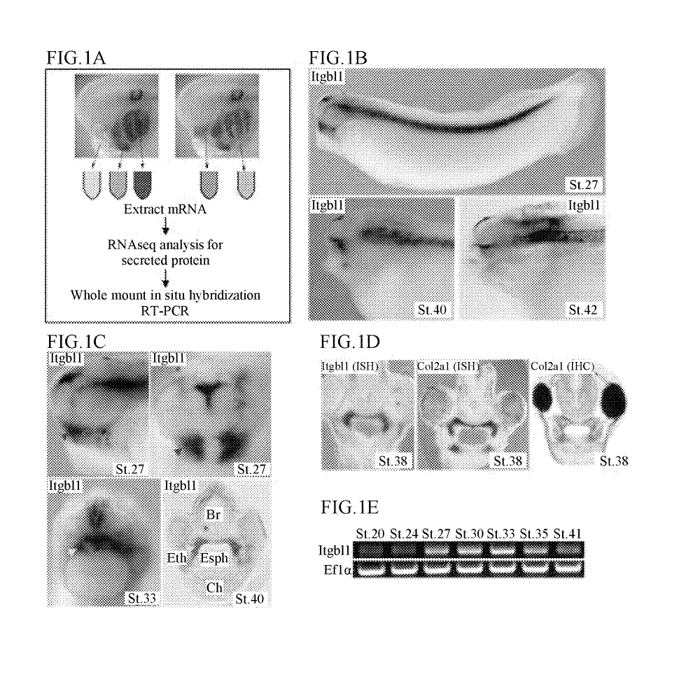

A result of the above analysis is shown in FIG. 1A, and analysis results are shown in Table 1 below.

TABLE 1

| SeqID | log2FC | Arch1 | Arch2 | Arch3 | ArchD | ArchV |
|---|---|---|---|---|---|---|
| LOC100491886.L\|HS=TLL2\|40\|Xelaev18021897m | 7.943 | 32.731 | 0.133 | 0.238 | 2.657 | 4.3 |
| sst.S\|HS=SST\|100\|Xelaev18029613m | 7.869 | 43.723 | 0.611 | 0.187 | 1.929 | 8.099 |
| clec19a.S\|HS=CLEC19A\|93\|Xelaev18047527m | 7.825 | 0.012 | 0.073 | 2.722 | 0.013 | 1.884 |
| fstl3.L\|HS=FSTL3\|86\|Xelaev18006463m | 7.311 | 0.065 | 1.509 | 10.318 | 0.383 | 8.008 |
| LOC100493098.L\|HS=DNASE1L2\|94\|Xelaev18045463m | 7.21 | 5.923 | 0.04 | 0.124 | 0.138 | 1.051 |
| unnamed\|HS=CA14\|77\|Xelaev18042787m | 7.102 | 10.163 | 0.425 | 0.074 | 0.131 | 0.89 |
| unnamed\|HS=HGFAC\|84\|Xelaev18005068m | 6.664 | 0.179 | 0.184 | 18.148 | 19.627 | 14.491 |
| unnamed\|HS=C4orf48\|46\|Xelaev18005037m | 6.647 | 3.908 | 0.111 | 0.039 | 0.24 | 0.179 |
| unnamed\|HS=FETUB\|74\|Xelaev18029648m | 6.635 | 0.115 | 1.254 | 11.427 | 46.345 | 10.328 |
| unnamed\|HS=FETUB\|66\|Xelaev18027506m | 6.349 | 0.144 | 1.23 | 11.735 | 56.671 | 8.782 |
| dct.L\|HS=DCT\|97\|Xelaev18013152m | 6.202 | 4.713 | 0.196 | 0.064 | 3.466 | 0.307 |
| tyr.S\|HS=TYR\|100\|Xelaev18016340m | 6.135 | 2.178 | 0.048 | 0.031 | 1.507 | 0.117 |
| unnamed\|HS=TLL2\|42\|Xelaev18021894m | 5.989 | 26.165 | 0.608 | 0.412 | 1.347 | 3.602 |
| fgfbp3.L\|HS=FGFBP3\|86\|Xelaev18034766m | 5.668 | 6.202 | 0.122 | 0.593 | 0.229 | 0.735 |
| c17orf67.L\|HS=C17orf67\|80\|Xelaev18043803m | 5.557 | 2.543 | 0.253 | 0.054 | 0.544 | 0.415 |
| serpinc1.L\|HS=SERPINC1\|93\|Xelaev18023232m | 5.493 | 0.746 | 2.766 | 33.603 | 28.227 | 27.363 |
| unnamed\|HS=C4orf48\|46\|Xelaev18008874m | 5.407 | 3.395 | 0.228 | 0.08 | 0.352 | 0.233 |
| Xetrov90010415m.S\|HS=NA\|00\|Xelaev18024389m | 5.366 | 0.079 | 3.257 | 2.948 | 1.784 | 3.945 |
| LOC101733976.S\|HS=EDN3\|55\|Xelaev18046257m | 5.293 | 6.859 | 0.175 | 0.198 | 1.025 | 1.698 |
| unnamed\|HS=CRISP3\|77\|Xelaev18028198m | 5.239 | 2.115 | 0.056 | 0.19 | 0.222 | 0.451 |
| unnamed\|HS=AGR3\|85\|Xelaev18044817m | 5.171 | 31.098 | 5.1 | 0.863 | 2.592 | 19.559 |
| gbp6.1\|HS=GBP1\|70\|Xelaev18001671m | 5.037 | 0.095 | 0.636 | 3.119 | 0.55 | 2.308 |
| LOC100490489.L\|HS=NA\|00\|Xelaev18027216m | 4.89 | 7.381 | 0.756 | 0.249 | 0.983 | 0.864 |
| pcdh8.2.S\|HS=PCDH8\|70\|Xelaev18015747m | 4.856 | 0.334 | 2.276 | 9.672 | 3.254 | 6.536 |
| npb.L\|HS=NPB\|84\|Xelaev18043747m | 4.783 | 3.552 | 0.285 | 0.129 | 0.153 | 0.208 |
| slurp1l.L\|HS=NA\|00\|Xelaev18023882m | 4.659 | 5.888 | 0.233 | 0.287 | 0.34 | 1.482 |
| LOC100485272-like.S\|HS=ANGPT2\|97\|Xelaev18015134m | 4.625 | 0.169 | 2.106 | 4.171 | 0.465 | 4.39 |
| Xetrov90021952m.L\|HS=TLL2\|43\|Xelaev18040194m | 4.555 | 27.59 | 1.174 | 3.595 | 1.248 | 5.305 |
| gdf7.L\|HS=GDF6\|85\|Xelaev18028105m | 4.507 | 0.239 | 1.846 | 5.436 | 6.902 | 0.795 |
| pmel-like.S\|HS=PMEL\|58\|Xelaev18015875m | 4.109 | 22.369 | 4.832 | 1.296 | 7.076 | 4.256 |
| tyrp1.L\|HS=TYRP1\|97\|Xelaev18006806m | 4.076 | 3.525 | 0.562 | 0.209 | 2.119 | 0.427 |
| vwde.L\|HS=VWDE\|78\|Xelaev18030909m | 3.993 | 4.473 | 1.18 | 0.281 | 0.756 | 1.698 |
| mcam-like.1\|HS=MCAM\|96\|Xelaev18000065m | 3.986 | 1.822 | 28.877 | 4.827 | 12.698 | 5.902 |
| sfrp1.L\|HS=SFRP1\|88\|Xelaev18018658m | 3.951 | 3.629 | 14.22 | 56.137 | 21.051 | 53.337 |
| apoc1-like.L\|HS=NA\|00\|Xelaev18036025m | 3.926 | 396.083 | 124.121 | 26.058 | 112.828 | 124.842 |
| mxra5.L\|HS=MXRA5\|43\|Xelaev18011748m | 3.876 | 3.993 | 1.212 | 0.272 | 0.62 | 1.325 |
| unnamed\|HS=TLL1\|44\|Xelaev18021890m | 3.871 | 80.83 | 17.585 | 5.526 | 18.007 | 37.359 |
| fstl3.S\|HS=FSTL3\|87\|Xelaev18009939m | 3.871 | 0.41 | 1.178 | 6 | 0.503 | 5.87 |
| hebp2.L\|HS=HEBP2\|79\|Xelaev18026611m | 3.862 | 7.083 | 0.976 | 0.487 | 0.71 | 1.197 |
| apoe.L\|HS=APOE\|85\|Xelaev18036024m | 3.792 | 53.004 | 16.025 | 3.827 | 5.81 | 16.975 |
| slc8a1-like.S\|HS=SLC8A1\|100\|Xelaev18028910m | 3.791 | 0.553 | 0.749 | 7.653 | 0.558 | 6.617 |
| cpn1.S\|HS=CPN1\|87\|Xelaev18037048m | 3.728 | 20.386 | 2.279 | 1.539 | 6.064 | 4.589 |
| LOC100496022.1\|HS=PRSS27\|92\|Xelaev18001642m | 3.708 | 21.708 | 7.858 | 1.661 | 3.825 | 13.988 |
| c8b.L\|HS=C8B\|93\|Xelaev18023028m | 3.61 | 0.502 | 1.028 | 6.13 | 14.667 | 7.128 |
| unnamed\|HS=RBP4\|89\|Xelaev18037044m | 3.608 | 17.786 | 15.571 | 1.459 | 9.927 | 4.092 |
| itga7-like.L\|HS=ITGA7\|95\|Xelaev18013342m | 3.573 | 0.694 | 0.799 | 8.258 | 0.479 | 5.348 |
| lrrn4.S\|HS=LRRN4\|97\|Xelaev18028812m | 3.506 | 0.337 | 0.967 | 3.828 | 1.595 | 5.497 |
| LOC100487362.L\|HS=LY9\|32\|Xelaev18040478m | 3.505 | 2.838 | 1.656 | 0.25 | 0.178 | 0.367 |
| rspo2.L\|HS=RSPO2\|100\|Xelaev18032283m | 3.424 | 6.911 | 2.565 | 0.644 | 1.476 | 1.476 |
| pyy.S\|HS=NPY\|96\|Xelaev18046061m | 3.418 | 0.355 | 0.218 | 2.33 | 0.794 | 0.257 |
| f2.S\|HS=F2\|98\|Xelaev18024485m | 3.414 | 0.39 | 0.933 | 4.157 | 4.675 | 3.267 |
| sel1l3.S\|HS=SEL1L3\|95\|Xelaev18008974m | 3.29 | 2.298 | 0.685 | 0.235 | 0.336 | 1.208 |
| unnamed\|HS=LCN15\|99\|Xelaev18038205m | 3.213 | 19.124 | 3.417 | 2.063 | 4.538 | 6.52 |
| unnamed\|HS=ANGPT1\|101\|Xelaev18032282m | 3.155 | 0.28 | 0.597 | 2.494 | 1.191 | 1.104 |
| nrn1.S\|HS=NRN1\|75\|Xelaev18033414m | 3.09 | 2.291 | 0.584 | 0.269 | 0.787 | 0.153 |
| fibin.S\|HS=FIBIN\|100\|Xelaev18024379m | 3.049 | 1.484 | 12.282 | 3.127 | 6.681 | 3.054 |
| olfml2a.S\|HS=OLFML2A\|96\|Xelaev18041818m | 3.046 | 7.192 | 1.606 | 0.871 | 1.836 | 2.419 |
| tgfb2.S\|HS=TGFB2\|100\|Xelaev18028671m | 3.035 | 1.73 | 6.055 | 14.175 | 6.469 | 18.302 |
| unnamed\|HS=GP1BB\|57\|Xelaev18010547m | 3.032 | 0.354 | 0.441 | 2.895 | 3.358 | 2.148 |
| unnamed\|HS=TECPR1\|7\|Xelaev18036521m | 3 | 4.055 | 2.121 | 0.507 | 0.423 | 2.221 |
| LOC100489571.S\|HS=MMP8\|97\|Xelaev18016259m | 2.96 | 1.139 | 8.863 | 4.084 | 0.027 | 3.147 |
| itgbl1.S\|HS=ITGBL1\|95\|Xelaev18015636m | 2.952 | 2.569 | 0.842 | 0.332 | 0.565 | 0.789 |
| unnamed\|HS=SCN4B\|90\|Xelaev18035439m | 2.871 | 0.958 | 2.188 | 7.008 | 2.412 | 5.981 |
| unnamed\|HS=TMEM213\|58\|Xelaev18002504m | 2.865 | 3.455 | 13.317 | 25.166 | 26.303 | 16.039 |
| unnamed\|HS=TNNT3\|67\|Xelaev18024361m | 2.857 | 1.944 | 6.248 | 14.083 | 4.678 | 8.964 |
| unnamed\|HS=CRISP3\|79\|Xelaev18030177m | 2.852 | 6.094 | 1.635 | 0.844 | 1.303 | 2.18 |
| unnamed\|HS=ANGPTL5\|64\|Xelaev18021227m | 2.835 | 5.622 | 1.554 | 0.788 | 0.778 | 1.391 |
| unnamed\|HS=LOXL4\|96\|Xelaev18034711m | 2.823 | 2.066 | 0.468 | 0.292 | 0.698 | 0.539 |
| prrt3-like.L\|HS=PRRT3\|67\|Xelaev18024053m | 2.819 | 3.727 | 1.313 | 0.528 | 0.91 | 0.589 |
| Xetrov90018420m.1\|HS=ROBO4\|99\|Xelaev18003883m | 2.79 | 2.325 | 3.695 | 16.082 | 8.331 | 10.299 |
| Xetrov90024887m.L\|HS=NA\|00\|Xelaev18043270m | 2.786 | 6.387 | 1.799 | 0.926 | 0.743 | 2.384 |
| fgf3.S\|HS=FGF3\|78\|Xelaev18024490m | 2.763 | 4.625 | 0.833 | 5.655 | 8.273 | 3.668 |
| unnamed\|HS=APELA\|100\|Xelaev18003936m | 2.747 | 3.911 | 8.978 | 26.249 | 11.39 | 22.66 |
| Xetrov90018420m.L\|HS=ROBO1\|17\|Xelaev18035307m | 2.712 | 2.853 | 3.26 | 18.696 | 13.616 | 8.351 |
| nov.S\|HS=NOV\|88\|Xelaev18033972m | 2.691 | 0.99 | 3.13 | 6.393 | 6.832 | 2.166 |
| unnamed\|HS=NELL2\|93\|Xelaev18017595m | 2.688 | 3.539 | 0.549 | 1.623 | 1.913 | 1.051 |
| cdh15.S\|HS=CDH15\|96\|Xelaev18024865m | 2.654 | 6.662 | 15.01 | 2.384 | 5.413 | 7.229 |
| igdcc3.L\|HS=IGDCC3\|89\|Xelaev18018436m | 2.64 | 4.418 | 2.005 | 0.709 | 2.334 | 1.009 |
| fgf8.L\|HS=FGF8\|79\|Xelaev18034665m | 2.609 | 19.454 | 3.188 | 9.32 | 11.654 | 11.125 |
| unnamed\|HS=ATP6AP1\|92\|Xelaev18017668m | 2.608 | 0.643 | 1.909 | 3.919 | 4.359 | 2.667 |
| c6.2.L\|HS=C6\|100\|Xelaev18008334m | 2.588 | 0.925 | 1.463 | 5.56 | 6.348 | 4.378 |
| unnamed\|HS=CA4\|90\|Xelaev18010514m | 2.569 | 2.391 | 0.566 | 0.403 | 0.673 | 0.462 |
| clec19a.L\|HS=CLEC19A\|83\|Xelaev18045207m | 2.558 | 0.525 | 1.552 | 3.091 | 0.9 | 2.85 |
| unnamed\|HS=CACNA2D4\|93\|Xelaev18003010m | 2.556 | 1.986 | 1.461 | 8.592 | 6.055 | 3.817 |
| unnamed\|HS=NELL2\|93\|Xelaev18021119m | 2.541 | 2.218 | 0.381 | 0.39 | 1.124 | 0.573 |
| unnamed\|HS=KDR\|96\|Xelaev18009068m | 2.525 | 1.047 | 5.879 | 6.025 | 3.959 | 7.705 |
| fam132a.S\|HS=FAM132A\|107\|Xelaev18037500m | 2.514 | 0.426 | 0.895 | 2.433 | 0.411 | 2.035 |
| LOC101733976.L\|HS=EDN3\|53\|Xelaev18043542m | 2.449 | 2.544 | 1.992 | 0.466 | 2.728 | 1.195 |
| nog2.S\|HS=NOG\|95\|Xelaev18047671m | 2.448 | 2.93 | 1.76 | 0.537 | 0.86 | 0.411 |
| npr3-like.L\|HS=NPR3\|90\|Xelaev18008385m | 2.429 | 8.499 | 3.773 | 1.578 | 2.862 | 2.916 |
| unnamed\|HS=LY86\|82\|Xelaev18031614m | 2.396 | 2.379 | 0.482 | 0.452 | 0.929 | 0.547 |
| Xetrov90000623m.L\|HS=SHISA3\|93\|Xelaev18005242m | 2.381 | 7.436 | 6.743 | 1.428 | 1.119 | 4.449 |
| ramp2.L\|HS=RAMP2\|61\|Xelaev18043825m | 2.377 | 1.44 | 3.696 | 7.482 | 5.008 | 3.22 |
| igfbpl1.L\|HS=IGFBPL1\|74\|Xelaev18006653m | 2.372 | 4.945 | 8.355 | 1.614 | 6.356 | 4.638 |
| stc2.L\|HS=STC2\|104\|Xelaev18017248m | 2.37 | 42.763 | 18.002 | 8.272 | 11.249 | 12.744 |
| LOC100496170-like.1\|HS=IZUMO1R\|68\|Xelaev18000799m | 2.37 | 11.898 | 2.97 | 2.301 | 3.944 | 4.662 |
| prtn3-like.1.L\|HS=PRTN3\|85\|Xelaev18006264m | 2.367 | 0.524 | 1.797 | 2.704 | 0.556 | 1.014 |
| fgfbp2.L\|HS=FGFBP2\|99\|Xelaev18005148m | 2.359 | 15.019 | 14.741 | 2.928 | 0.936 | 12.958 |
| unnamed\|HS=CFI\|105\|Xelaev18005652m | 2.32 | 3.237 | 7.08 | 16.167 | 28.023 | 12.637 |
| mmp11.S\|HS=MMP11\|92\|Xelaev18010511m | 2.316 | 3.304 | 2.639 | 13.137 | 1.768 | 11.177 |

Also, to determine whether ITGBL1 is expressed in chondrocytes of Xenopus laevis embryos, the following experiment was conducted.

An RNA probe having a base sequence complementary to ITGBL1 mRNA was prepared using an Ambion Megascript T7 RNA Transcription kit (Promega, P2077), and craniofacial tissue slices of Xenopus laevis embryos at stage 37 that were dissected as described above and fixed, were treated with a prehybridization buffer (formamide 1.13 g/ml, Biosesang, F1014 in 2×SSC; 0.3 M NaCl-Biosesang, 30 mM sodium citrate Biosesang, C1029) at 65° C. for 12 hours. The treated craniofacial tissue slices were washed twice with a 2×SSC solution at 65° C. for 30 minutes, were treated with RNase, were washed twice with a 0.2×SSC solution for 30 minutes, were treated with an anti-digoxigenin antibody (Sigma) for 12 hours and were washed three times with TBST. A color reaction was induced using BM purple (Sigma).

Sequences of primers to prepare the RNA probe are shown in Table 2 below.

TABLE 2

| Name | Sequence (5′ → 3′) | SEQ ID NO: |
|---|---|---|
| ITGBL1 5′ | augcacgcuggagccuu | 1 |
| ITGBL1 3′ | aggauauucgcuuccaagcca | 2 |

Analysis results are shown in FIGS. 1B and 1C.

As shown in FIGS. 1B and 1C, it is confirmed that ITGBL1 is expressed in chondrocytes of Xenopus laevis embryos.

1-2. Comparison Between Expression Pattern of Col2a1 and Expression Pattern of ITGBL1

Fixed Xenopus laevis embryos at stage 37 were cross-sectioned at a thickness of 100 μm using a Vibratome (Leica, VT 1000S). An RNA probe having a base sequence complementary to ITGBL1 mRNA, and an RNA probe having a base sequence complementary to Col2a1 mRNA were prepared, and craniofacial tissue slices of the fixed Xenopus laevis embryos at stage 37 were treated with a prehybridization buffer (formamide 1.13 g/ml, Biosesang, F1014) (w/v) at 65° C. for 12 hours. Each of the prepared RNA probes were diluted in a prehybridization solution, and the craniofacial tissue slices were treated at 65° C. for 1 day. The treated craniofacial tissue slices were washed twice with a 2×SSC solution (0.3 M NaCl-Biosesang, 20 mM sodium citrate-Biosesang, C1029) at 65° C. for 30 minutes, were treated with RNase, were washed twice with a 0.2×SSC solution for 30 minutes, were treated with an anti-digoxigenin antibody (Sigma) for 12 hours, and were washed three times with TBST. A color reaction was induced using BM purple (Sigma). In another method, a DAB staining, that is, an immunostaining of treating a slice with a thickness of 100 μm with a Col2a1 antibody (DSHB, II-II6B3) to confirm an cartilage-specific matrix expression, was conducted.

As shown in FIG. 1D, it is confirmed that ITGBL1 is expressed in cartilage tissues because an expression pattern of Col2a1 and an expression pattern of ITGBL1 are similar to each other.

1-3. Confirmation of ITGBL1 Expression Time

RNA was extracted from fertilized Xenopus laevis embryos at stages 20, 24, 27, 30, 33, 35 and 41 using a PureLink RNA Mini Kit (Invitrogen, 12183018A), and cDNA was synthesized using GoScript Reverse Transcriptase (Promega, A5004A). Reverse transcription polymerase chain reaction (RT-PCR; BIO RAD, T100 Thermal Cycler) was performed on the synthesized cDNA using Taq polymerase (Coregen, CE-500U).

As shown in FIG. 1E, it is confirmed that ITGBL1 is most strongly expressed at stage 30 that is a period in which cartilage tissues are formed.

### EXAMPLE 2

### ITGBL1's Function of Promoting Formation of Cartilage Tissues

2-1. Case of Inhibiting ITGBL1 Expression

To confirm a function of ITGBL1 to promote a formation of cartilage tissues, expression of ITGBL1 was inhibited in the following manner

To use splice-blocking antisense morpholino oligonucleotides to inhibit expression of ITGBL1 in chondrocytes of Xenopus laevis embryos, a preparation of splice-blocking antisense morpholino oligonucleotides was requested to Gene Tools, LLC, and a sequence thereof is shown in Table 3 below.

TABLE 3

| Name | Sequence (5′→3′) | SEQ ID NO: |
|---|---|---|
| ITGBL1 MO | AGTAGGGAAGATATACAGACCTGCA | 3 |

The prepared splice-blocking antisense morpholino oligonucleotides were injected into dorsal-ventral axis of Xenopus laevis embryos at 2-cell stage, to inhibit the ITGBL1 expression in chondrocytes. Embryos into which the splice-blocking antisense morpholino oligonucleotides were injected were cultured up to embryo stage 45, and fixed with MEMFA (4% Formaldehyde, Biosesang, F1012). A specific staining of cartilage was performed using Alcian blue (1% Alcian Blue 8GX, Georgiachem, AB1082) and tissues other than the cartilage were removed using trypsin, to remove a nonspecific staining using a 10% (v/v) methanol solution. The cartilage was observed using an Olympus (SZX16-ILLB) microscope.

As a control group, an experiment was conducted in the same manner as described above, except that morpholino oligonucleotides that inhibit expression of ITGBL1 in chondrocytes of Xenopus laevis embryos were not injected.

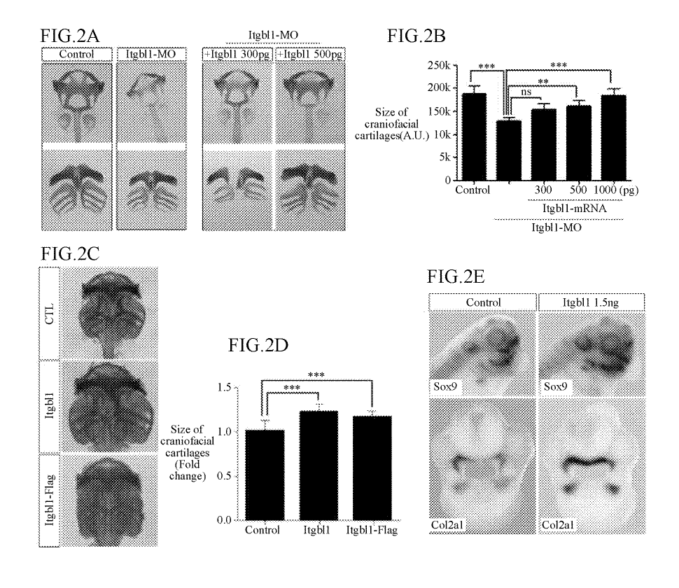

Results of the experiment are shown in FIGS. 2A and 2B.

As shown in FIGS. 2A and 2B, it is confirmed that when the expression of the ITGBL1 in the chondrocytes of the Xenopus laevis embryos is inhibited, a size and shape of cartilage are reduced abnormally in comparison to the control group in which expression of ITGBL1 is not inhibited.

2-2. Case of Increasing ITGBL1 Expression

To confirm a function of ITGBL1 to promote a formation of cartilage tissues, expression of ITGBL1 was increased in the following manner

ITGBL1 cDNA (SEQ ID NO: 4) obtained from facial chondrocytes of Xenopus laevis embryos was applied to an mMESSAGE mMACHINE SP6 kit (Ambion, AM1340), to synthesize ITGBL1 mRNA. The ITGBL1 mRNA was injected into Xenopus laevis embryos in the same manner as in Example 2-1, to increase expression of ITGBL1 in chondrocytes. An Alcian blue staining was performed in the same manner as in Example 2-1, and expression of Sox9 and Col2a1 was confirmed through whole-mount in situ hybridization (WISH).

As a control group, an experiment was conducted in the same manner as described above, except that ITGBL1 mRNA that increases expression of ITGBL1 in chondrocytes of Xenopus laevis embryos was not injected.

Results of the experiments are shown in FIGS. 2C, 2D and 2E.

As shown in FIGS. 2C and 2D, it is confirmed that when the expression of the ITGBL1 is increased by injecting the ITGBL1 mRNA into the facial chondrocytes of the Xenopus laevis embryos, a size of cartilage is increased 1.2 times to 1.3 times in comparison to the control group in which the expression of the ITGBL1 is not increased. As shown in FIG. 2E, it is confirmed that expression of Sox9 and Col2a1 that are markers of cartilage tissues is increased in comparison to the control group.

### EXAMPLE 3

### Effect of Promoting Chondrogenesis by ITGBL1 Protein in Human and Mouse Chondrocytes

3-1. Comparison of ITGBL1 Expression between Chondrocytes and Bone Tissues During a Differentiation of BM-MSCs

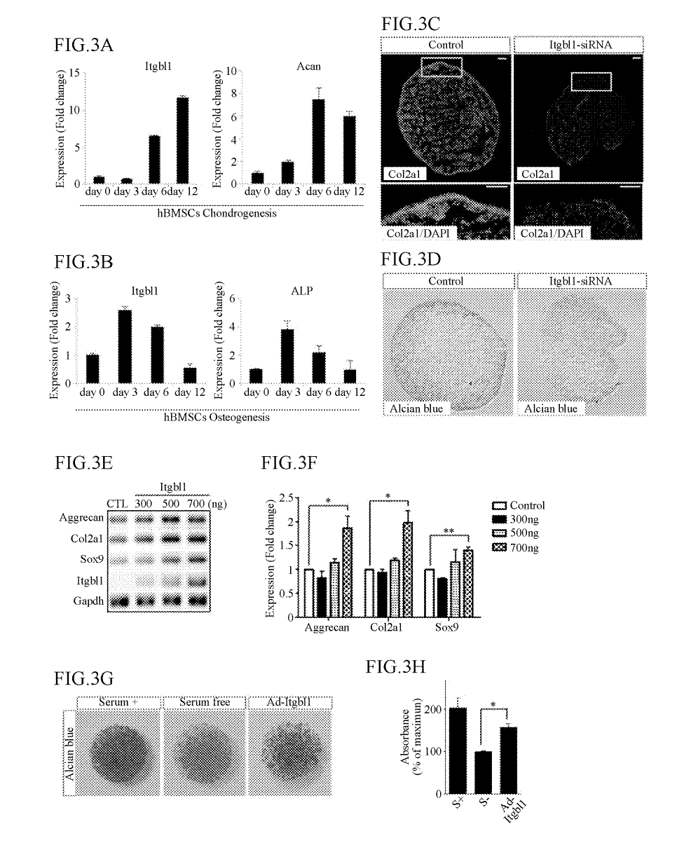

To determine whether chondrogenesis is promoted by an ITGBL1 protein during a differentiation of human bone marrow-derived mesenchymal stem cells (hBMSCs), an experiment was conducted in the following manner hBMSCs (ATCC) were cultured in an α-minimal essential medium (α-MEM; Welgene, LM008-01, containing 10% (v/v) FBS and 1% (v/v) antibiotics). After forming a cell mass using a micromass method, the cell mass was treated with a chondrogenic inducer (TGF-β, dexamethasome, ascorbate-2-phosphate) together with the culture solution, to induce a formation of cartilage tissues. The formation of the cartilage tissues was induced for 12 days, and RNA was extracted using a PureLink RNA Mini Kit (Invitrogen, 12183018A), to synthesize cDNA using a GoScript Reverse Transcriptase (Promega, A5004A). Quantitative Reverse Transcription (qRT)-PCR was performed on the synthesized cDNA using a QuantStudio™ 6 Flex Real-Time PCR System (Applied Biosystems, Foster City, Calif., USA). All reactions were performed on 96-well plates, and a mean value was used to calculate mRNA expression. Results of the experiment are shown in FIGS. 3A and 3B.

As shown in FIG. 3A, it is confirmed that expression of ITGBL1 increases during a differentiation into chondrocytes of hBMSCs. As shown in FIG. 3B, it is confirmed that expression of ITGBL1 decreases during a formation of bone tissues of the hBMSCs.

3-2. Case of Inhibiting ITGBL1 Expression During Differentiation into Chondrocytes of hBMSCs and Case of Increasing ITGBL1 Expression in Chondrocytes

To determine whether chondrogenesis is promoted by an ITGBL1 protein in human, an experiment was conducted in the following manner

hBMSCs (ATCC) were cultured in an α-minimal essential medium (α-MEM; Welgene, LM008-01, containing 10% (v/v) FBS and 1% (v/v) antibiotics), and ITGBL1 siRNA (Genolution, SEQ ID NO: 5 and 6) were transfected into the hBMSCs using a method of a protocol provided by a manufacturer. After forming a cell mass using a micromass method, the cell mass was treated with a chondrogenic inducer (TGF-β, dexamethasome, ascorbate-2-phosphate) together with the culture solution, to induce a formation of cartilage tissues. After the formation of the cartilage tissues was induced for 7 days, the cartilage tissues were fixed through a treatment with 4% (w/v) paraformaldehyde. The fixed cartilage tissues were added to an OCT solution (Cell Path, KMA-0.00-00A) and were cut at a thickness of 15 μm using a cryotome (Bright, OTF5000), and an immunofluorescent staining was performed to confirm expression of Col2a1. To determine whether chondrogenesis is promoted, an Alcian blue staining was performed in the same manner as in Example 2-1.

In the case of inhibiting expression of ITGBL1, ITGBL1 siRNA was not transfected in a control group. To increase ITGBL1 expression in chondrocytes, human chondrocytes (Cell Application, Inc. San Diego, Calif., USA) were cultured in an α-MEM (Welgene, LM008-01, containing 10% (v/v) FBS and 1% (v/v) antibiotics), and ITGBL1 cDNA (SEQ ID NO: 7, ITGBL1 protein SEQ ID NO: 8) were transfected into the human chondrocytes using a method of a protocol provided by a manufacturer. The cell mass was treated with a chondrogenic inducer (TGF-β, dexamethasome, ascorbate-2-phosphate) together with the culture solution, to induce a formation of cartilage tissues.

After the formation of the cartilage tissues was induced for 7 days, total RNA was extracted from chondrocytes in which ITGBL1 DNA was transfected, using a PureLink RBA Mini Kit (Invitrogen, 12183018A), cDNA was synthesized using GoScript Reverse Transcriptase (Promega, A5004), and qRT-PCR was performed using a QuantStudio™ 6 Flex Real-Time PCR System (Applied Biosystems, Foster City, Calif., USA). All reactions were performed on 96-well plates, and a mean value was used to calculate mRNA expression. Sequences of ITGBL1 siRNA and primers used in the qRT-PCR are shown in Table 4 below.

TABLE 4

| Name | Sequence (5′→3′) | Tm (° C.) | SEQ ID NO: |
|---|---|---|---|
| Human | Sense: GAGCUGUCUAUGACCGAUAUU | N/A | 5, 6 |
| ITGBL1- | Antisense: UAUCGGUCAUAGACAGCUCUU | | |
| siRNA | | | |
| Mouse | ATGCATCCTCCAGGCTTCAGGAACTTCTT | N/A | 7 |
| ITGBL1 | GTTGCTGGTGTCCTCCCTTCTCTTCATTGG | | |
| cDNA | GCTGTCAGCTGCTCCTCAAAGCTTCTTAC | | |
| | CATCTCTGAGAAGCCTGTCGGGCGCCCCC | | |
| | TGCAGGCTGTCCCGGGCAGAGTCCGAAC | | |
| | GCAGATGTCGTGCACCTGGGCAGCCCCC | | |
| | AGGGAGCGCTCTGTGCCATGACCGTGGC | | |
| | CGGTGCGAGTGTGGGGTCTGCATCTGTCA | | |
| | CGTGACCGAACCTGGCACCTACTTCGGTC | | |
| | CACTGTGTGAGTGCCATGAGTGGATATG | | |
| | CGAGACCTACGACGGGAAAACCTGTGCA | | |
| | GGCCACGGTAATTGTGACTGCGGCAAGT | | |
| | GCAAGTGTGATGTGGGATGGTCTGGGGA | | |
| | AGCTTGTCAGTACCCAACCAAGTGTGAC | | |
| | CTGACCAAAAAAATCAGCAACCAGATGT | | |
| | GCAAGAACTCCCAAGATGTCATCTGCTCC | | |
| | AATGCAGGTACATGTCACTGTGGCAGGT | | |
| | GTAAGTGTGATAATTCAGATGGACATGG | | |
| | ACTCATTTATGGTAAATTTTGTGAATGTG | | |
| | ATGATAGAGAATGCATAGATGATGAAAC | | |
| | AGAAGAAGTATGTGGAGGCCATGGGAAG | | |
| | TGTTACTGTGGAAACTGTTACTGTGAGGC | | |
| | TGGTTGGCATGGCGATAAATGCGAGTTC | | |
| | CAGTGTGACATCACCCCATGGGAAAGCA | | |
| | AGCGAAGATGCACATCTCCAGATGGCAA | | |
| | AGTCTGTAGCAACAGAGGAACATGTGTA | | |
| | TGTGGTGAATGTTCTTGCCATGATGTTGA | | |
| | TCCAACTGGGGACTGGGGAGACATTCAT | | |
| | GGGGACACGTGTGAGTGTGATGAAAGGG | | |
| | ACTGCAGAGCTGTTTATGATCGATACTCT | | |
| | GATGATTTCTGTTCAGGTCATGGGCAGTG | | |
| | TAACTGTGGAAGATGTGACTGCAGAGCA | | |
| | GGCTGGTATGGGAAGAAATGTGAGCACC | | |
| | CAAGGAATTGCCCATTGTCAGCTGAGGA | | |
| | GAGCACCAAGAAGTGCCAGGGTAGTTCT | | |
| | GATCTTCCTTGCTCTGGAAGGGGCAGATG | | |
| | CGAATGTGGCAGATGCACTTGTTACCCTC | | |
| | CTGGGGACAGCAGAGTCTATGGCAAGAC | | |
| | CTGTGAGTGTGATGACCGGCGCTGCGAG | | |
| | GACCTGGATGGTGTGGTCTGCGGAGGCC | | |
| | ATGGCATGTGCTCCTGTGGTCGCTGTGTT | | |
| | TGTGAGAAAGGATGGTTTGGTAAGCTCT | | |
| | GCCAACACCTGCGGAAGTGTAATATGAC | | |
| | AGAAGAACAAAGCAGGAGTCTGTGTGAG | | |
| | TCAGCAGATGGCACATTGTGCTCAGGGA | | |
| | AGGGTTCTTGTCATTGTGGAAAGTGCATT | | |
| | TGTTCTGGAGAAGAGTGGTATATTTCAGG | | |
| | GGAGTTTTGTGACTGTGATGACAGAGAC | | |
| | TGTGACAAACACGATGGTCTCATTTGCAC | | |
| | AGGGAATGGAATCTGTAGCTGTGGAAAC | | |
| | TGTGAATGCTGGGATGGATGGAATGGAA | | |
| | ATGCATGTGAAATCTGGCTTGGTACCGA | | |
| | ATATCCTTAA | | |
| Mouse | MHPPGFRNFLLLVSSLLFIGLSAAPQSFLPS | N/A | 8 |
| ITGBL1 | LRSLSGAPCRLSRAESERRCRAPGQPPGSA | | |
| Protein | LCHDRGRCECGVCICHVTEPGTYFGPLCEC | | |
| | HEWICETYDGKTCAGHGNCDCGKCKCDV | | |
| | GWSGEACQYPTKCDLTKKISNQMCKNSQD | | |
| | VICSNAGTCHCGRCKCDNSDGHGLIYGKF | | |
| | CECDDRECIDDETEEVCGGHGKCYCGNCY | | |
| | CEAGWHGDKCEFQCDITPWESKRRCTSPD | | |
| | GKVCSNRGTCVCGECSCHDVDPTGDWGDI | | |
| | HGDTCECDERDCRAVYDRYSDDFCSGHGQ | | |
| | CNCGRCDCRAGWYGKKCEHPRNCPLSAEE | | |
| | STKKCQGSSDLPCSGRGRCECGRCTCYPPG | | |
| | DSRVYGKTCECDDRRCEDLDGVVCGGHG | | |
| | MCSCGRCVCEKGWFGKLCQHLRKCNMTE | | |
| | EQSRSLCESADGTLCSGKGSCHCGKCICSG | | |
| | EEWYISGEFCDCDDRDCDKHDGLICTGNGI | | |
| | CSCGNCECWDGWNGNACEIWLGTEYP | | |
| SOX9-F | AAGGAGAGCGAGGAGGACAAGTTC | 62 | 9 |
| SOX9-B | TGTTCTTGCTGGAGCCGTTG | 57.4 | 10 |
| MMMP3-F | GATGCGCAAGCCCAGGTGTG | 64.13 | 11 |
| MMP3-B | GCCAATTTCATGAGCAGCAACGA | 59.57 | 12 |
| MMP13-F | AGGAGCATGGCGACTTCTACCC | 62.88 | 13 |
| MMP13-B | TTTGTCTGGCGTTTTTGGATGTTT | 56.23 | 14 |
| Col2a1-F | CAGTTGGGAGTAATGCAAG | 58 | 15 |
| Col2a1-B | GCCTTGAGCAGTTCACCTTC | 58 | 16 |

Results of the experiment are shown in FIGS. 3C, 3D and 3E.

As shown in FIGS. 3C and 3D, it is confirmed that when expression of an ITGBL1 protein is inhibited, a formation of cartilage tissues is suppressed.

As shown in FIGS. 3E and 3F, it is confirmed that expression of Sox9 and Col2a1 that are chondrogenic factors gradually increases when expression of an ITGBL1 protein gradually increases and that a formation of cartilage tissues is promoted.

Also, limb bud mesenchymes differentiating from arms and legs of mouse (limb bud) embryos into chondrocytes were isolated. An adenovirus vector (Ad-ITGBL1; Vector Biolabs, Malvern, Pa. 19355 USA, customized by a company) that contains ITGBL1 was transduced, to increase expression of ITGBL1 and induce a cartilage differentiation. It is confirmed that the cartilage differentiation is promoted using an Alcian blue staining scheme. Results of the experiment are shown in FIGS. 3G and 3H.

As shown in FIGS. 3G and 3H, in chondrocytes in which expression of ITGBL1 is increased, an amount of glycosaminoglycan (GAG) is increased and chondrogenesis is promoted.

### EXAMPLE 4

### Effect of ITGBL1 Protein to Promote Chondrogenesis in Arthritis-Induced Mice

To determine whether an ITGBL1 protein promotes chondrogenesis in arthritis-induced mice, an experiment was conducted in the following manner

Chondrocytes isolated from knee cartilage of postnatal day 5 mice were cultured for 2 days, and were treated with 5 ng/ml of IL-1β (GenScript, 201-LB) for 72 hours in order to induce an inflammation. An adenovirus vector Ad-ITGBL1 was transduced into the chondrocytes in which the inflammation was induced, and a change in expression of a chondrogenic factor (Sox9 and Col2a1) was analyzed by qRT-PCR in the same manner as in Example 3. Sequences of primers used in the qRT-PCR are shown in Table 5 below.

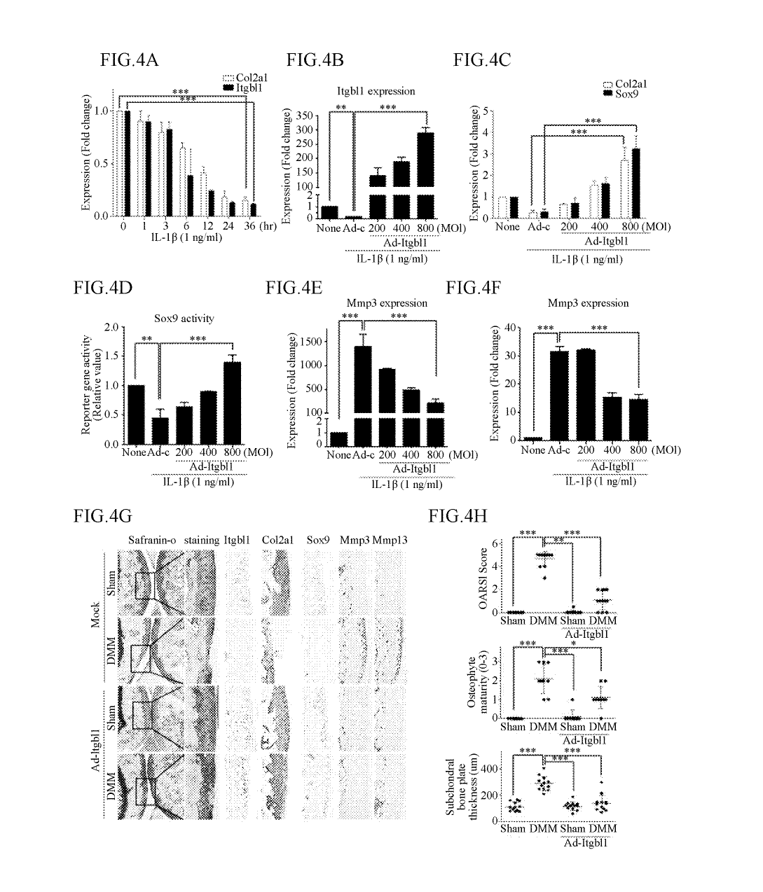

As a control group, an experiment was conducted in the same manner as described above, except that an adenovirus vector was not transduced and a treatment with IL-1β was not performed. The control group is indicated by “None” in FIGS. 4B through 4F.

TABLE 5

| | | Tm | SEQ ID |
|---|---|---|---|
| Name | Sequence (5′→3′) | (° C.) | NO: |
| Col2a1-F | CACACTGGTAAGTGGGGCAAGA | 55 | 21 |
| Col2a1-B | GGATTGTGTTGTTTCAGGGTTCG | 55 | 22 |
| SOX9-F | CATCAGCAGCACCGCACCCA | 58 | 23 |
| SOX9-B | CGGGTGATGGGCGGGTAGGA | 58 | 24 |

Results of the experiment are shown in FIGS. 4A through 4D.

As shown in FIG. 4A, when an inflammation is induced by treating chondrocytes isolated from mouse knee cartilage with IL-1β, expression of ITGBL1 is significantly reduced. As shown in FIGS. 4B through 4D, when expression of ITGBL1 is increased, expression of a chondrogenic factor (Sox9 and Col2a1) is increased.

Also, osteoarthritis (OA) was induced in a joint of a mouse by a destabilization of medial meniscus (DMM) surgery as a meniscectomy used to induce arthritis by removing medial meniscuses at knee joints of mice, and an adenovirus vector Ad-ITGBL1 was transduced into the joint of the mouse, to increase expression of an ITGBL1 protein. After 6 weeks, the joint of the mouse was isolated and stained with Safranin O, and expression levels of Col2a1 and Sox9 were compared using immunohistochemistry. Results of the experiment are shown in FIGS. 4G and 4H.

As a control group, an experiment was conducted in the same manner as described above, except that a meniscectomy was not performed. The control group is indicated by “Sham” in FIGS. 4G and 4H.

As shown in FIGS. 4G and 4H, it is confirmed that when expression of an ITGBL1 protein is increased in a joint of an arthritis-induced mouse, an arthritis score (Osteoarthritis Research Society International (OARSI)) is reduced and expression of Col2a1 and Sox9 is increased, thereby inhibiting arthritis.

### EXAMPLE 5

### Inflammation Inhibitory Effect of ITGBL1 Protein in Arthritis-Induced Mice

To confirm an inflammation inhibitory effect of an ITGBL1 protein in arthritis-induced mice, an experiment was conducted in the following manner

As described above in Example 4, mouse chondrocytes were treated with 5 ng/ml of IL-1β for 72 hours in order to induce an inflammation. An adenovirus vector containing ITGBL1 DNA (Ad-ITGBL1) was transduced into the mouse chondrocytes in which the inflammation was induced, and a change in expression of MMP3 and MMP13 that are inflammatory factors was analyzed using qRT-PCR in the same manner in Example 3. Sequences of primers used in the qRT-PCR are shown in Table 6 below.

As control groups, an experiment was conducted in the same manner as described above, except that an adenovirus vector into which ITGBL1 was not inserted was transduced, as indicated by “Mock” and that an adenovirus vector was not transduced and a treatment with IL-1β was not performed as indicated by “None”.

TABLE 6

| | | Tm | SEQ ID |
|---|---|---|---|
| Name | Sequence (5′→3′) | (° C.) | NO: |
| MMP3-F | TCCTGATGTTGGTGGCTTCAG | 58 | 17 |
| MMP3-B | TGTCTTGGCAAATCCGGTGTA | 58 | 18 |
| MMP13-F | ACCACATCGAACTTCGA | 58 | 19 |
| MMP13-B | CGACCATACAGATACTG | 58 | 20 |

Results of the experiment are shown in FIGS. 4E and 4F.

As shown in FIGS. 4E and 4F, it is confirmed that when expression of an ITGBL1 protein is increased in mouse chondrocytes in which arthritis is induced by a treatment with IL-1β, expression of an inflammatory factor (MMP3 and MMP13) is significantly inhibited.

Also, osteoarthritis (OA) was induced in a joint of a mouse by a destabilization of medial meniscus (DMM) surgery as a meniscectomy, and an adenovirus vector Ad-ITGBL1 was transduced into the joint of the mouse, to increase expression of an ITGBL1 protein. After 6 weeks, the joint of the mouse was dissected, and expression of MMP3 and MMP13 were examined by performing an immunohistochemistry staining. Results thereof are shown in FIGS. 4G and 4H.

As a control group, an experiment was conducted in the same manner as described above, except that a meniscectomy was not performed, as indicated by “Sham” in FIGS. 4G and 4H.

As shown in FIGS. 4G and 4H, it is confirmed that when expression of an ITGBL1 protein is increased in a joint of an arthritis-induced mouse, expression of MMP3 and MMP13 that are inflammatory factors is reduced and osteoarthritis does not develop as severe as control.

### EXAMPLE 6

### Effect of ITGBL1 Protein to Inhibit Integrin Activation

6-1. Analysis of Change in Focal Adhesion Complexes Based on Inhibition of ITGBL1 Expression

To confirm a change in focal adhesion complexes by an ITGBL1 protein, an experiment was conducted in the following manner

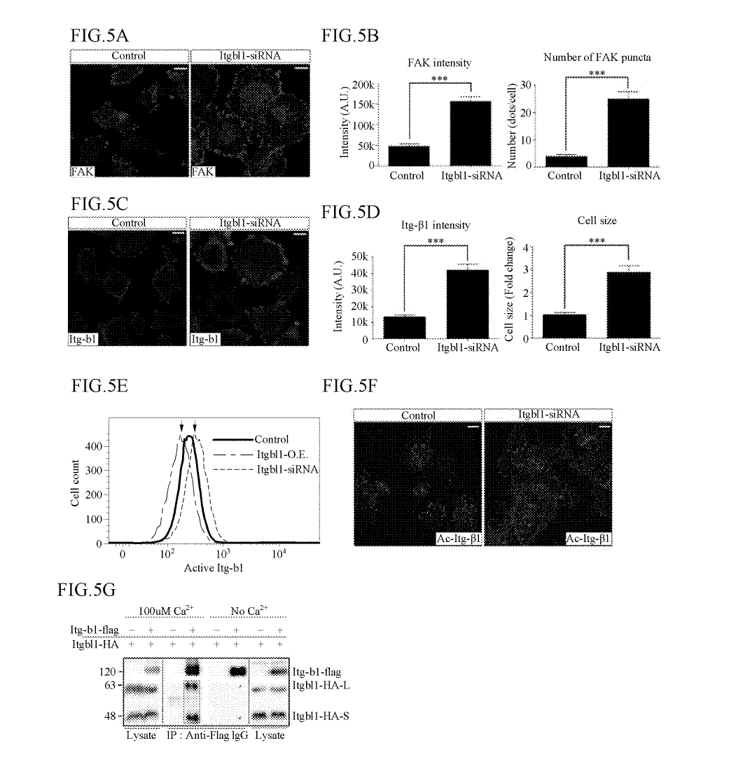

PC3 cells were cultured in an RPMI 1640 medium (gibco 22400-099, containing 10% (v/v) FBS and 1% (v/v) antibiotics), and 2×10⁵ cells were seeded on a 6-well plate and were transfected with ITGBL1 siRNA (Genolution 1) or ITGBL1 DNA using a jetPRIME (Polyplus, 114-15) based on a method of a protocol provided by a manufacturer. The transfected cells were detached from the bottom using 0.25% Trypsin EDTA (gibco, 25200-072), were attached onto a fibronectin-coated coverslip for 4 hours, and were fixed in 4% (w/v) paraformaldehyde. To find a change in focal adhesion complexes, an immunofluorescent staining analysis was performed using anti-FAK (abcam, ab40794) and anti-β1 integrin (DSHB, AIIB2). Results of the experiment are shown in FIGS. 5A through 5D.

As shown in FIGS. 5A through 5D, it is confirmed that an amount of focal adhesion complexes generated in a binding site of a cell matrix and integrin significantly increases when expression of ITGBL1 is inhibited in a PC3 cell line using ITGBL1-siRNA. 6-2. Analysis of Correlation Between ITGBL1 and Integrin Activation

To confirm a correlation between an ITGBL1 protein and integrin activation, the following experiment was conducted.

PC3 cells were cultured in an RPMI 1640 medium (gibco 22400-099, containing 10% (v/v) FBS and 1% (v/v) antibiotics), and 2×10⁵ cells were seeded on a 6-well plate and were transfected with ITGBL1 siRNA (Genolution 1) or ITGBL1 DNA using a jetPRIME (Polyplus, 114-15) based on a method of a protocol provided by a manufacturer. The transfected cells were detached from the bottom using 4 mM EDTA, an immunofluorescent staining was performed using an antibody (MILLIPORE, MAB2079Z) that binds to β1-integrin activated in a fluorescence-activated cell sorting (FACS) buffer (1× PBS, 2% FBS), and an analysis was performed using BD LSRFortessa™.

As shown in FIGS. 5E and 5F, it is confirmed that an amount of activated integrin increases when expression of ITGBL1 is inhibited, and that the amount of activated integrin decreases when expression of ITGBL1 is increased.

To confirm an interaction between ITGBL1 and integrin-β1, a co-immunoprecipitation experiment was conducted.

An ITGBL1-HA vector and an ITGB1-flag (integrin-β1) vector were transfected into HEK 293T cells, to extract cell proteins. The extracted cell proteins were subjected to the co-immunoprecipitation experiment using paramagnetic beads (Dynabeads, ThermoFisher) with an HA antibody. Results of the experiment are shown in FIG. 5G.

As shown in FIG. 5G, it is confirmed that ITGBL1 binds to ITGB1 in a state in which calcium ions are present.

6-3. Confirmation of Cell Adhesion and Integrin Activity Based on Increase in ITGBL1 Expression

PC3 cells were cultured in an RPMI 1640 medium (gibco 22400-099, containing 10% (v/v) FBS and 1% (v/v) antibiotics), chondrocytes were cultured in an α-MEM (Welgene, LM008-01, containing 10% (v/v) FBS and 1% (v/v) antibiotics), and 2×10⁵ cells were seeded on a 6-well plate and were transfected with ITGBL1 siRNA (Genolution 1) or ITGBL1 DNA using a jetPRIME (Polyplus, 114-15) based on a method of a protocol provided by a manufacturer. The transfected cells were detached from the bottom using 0.25% Trypsin EDTA (gibco, 25200-072), were attached onto a fibronectin-coated coverslip for 4 hours by treating the cells on the media based on a concentration of Mn²⁺, and were fixed in 4% (w/v) paraformaldehyde. The cells were observed using an Olympus IX73 microscope.

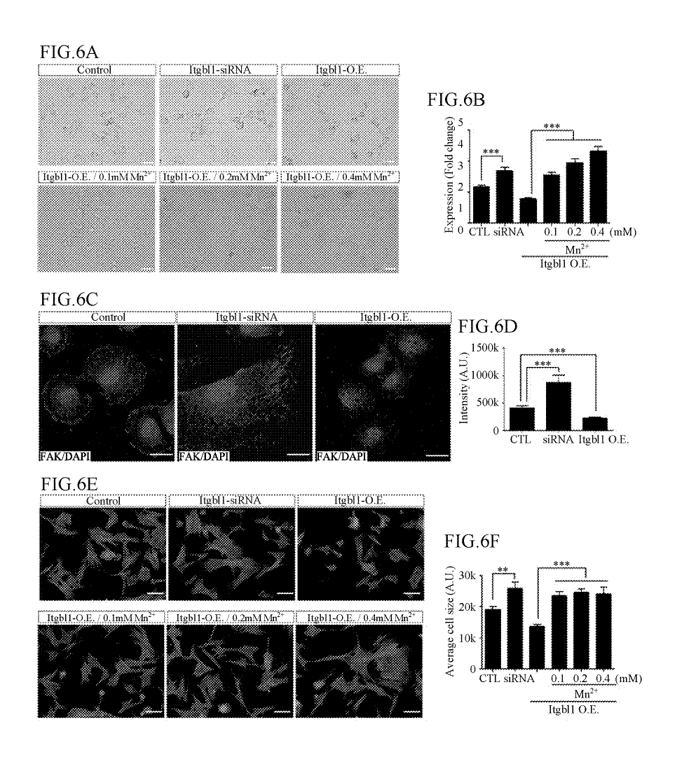

It is confirmed that when expression of ITGBL1 is increased in all of PC3 cells (FIGS. 6A and 6B), human mesenchymal stem cells (FIGS. 6C and 6D) and human chondrocytes (FIGS. 6E and 6F), a cell adhesion is inhibited, but that when integrin activation is increased by a treatment with Mn²⁺, the cell adhesion is increased again. Thus, it is demonstrated that the ITGBL1 protein inhibits the integrin activation to inhibit the cell adhesion.

### EXAMPLE 7

### Analysis of Correlation between Integrin Activation Inhibition Function of ITGBL1 Protein and Control of Expression of Chondrogenic Factor of Chondrocytes

7-1. Case of Increasing ITGBL1 Expression

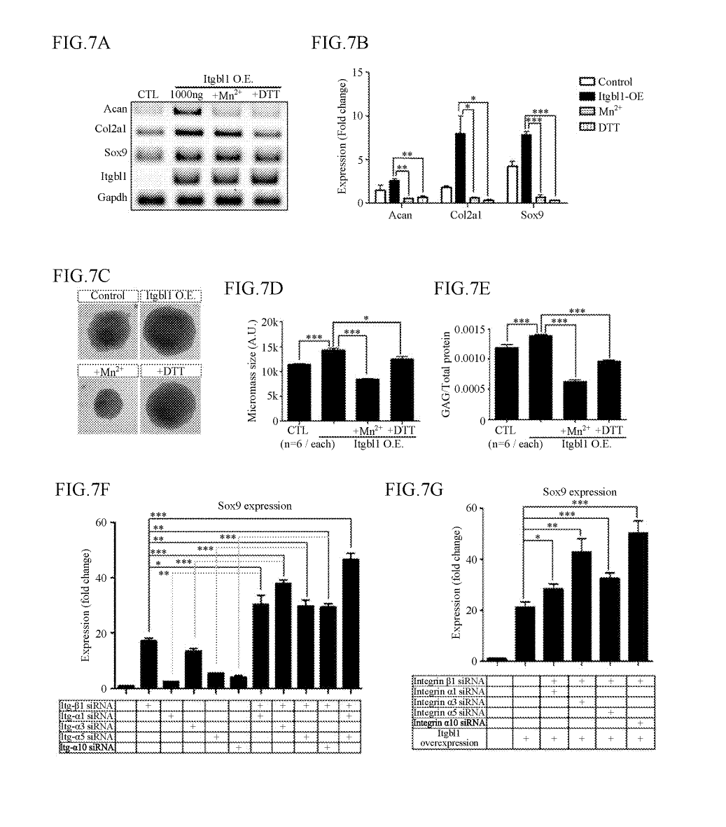

To analyze an influence of an integrin activation inhibition function of ITGBL1 on chondrogenesis during the chondrogenesis, the following experiment was conducted. Chondrocytes were cultured in an α-MEM (Welgene, LM008-01, containing 10% (v/v) FBS and 1% (v/v) antibiotics), and 2×10⁵ cells were seeded on a 6-well plate and were transfected with ITGBL1 DNA using a jetPRIME (Polyplus, 114-15) based on a method of a protocol provided by a manufacturer. The transfected chondrocytes were treated with Mn²⁺ or DTT and incubated at 37° C. RNA was extracted from the cells using a PureLink RNA Mini Kit (Invitrogen, 12183018A), and cDNA was synthesized using GoScript Reverse Transcriptase (Promega, A5004A). RT-PCR (BIO RAD, T100 Thermal Cycler) was performed on the synthesized cDNA using Taq polymerase (Coregen, CE-500U). Also, qRT-PCR was performed using a QuantStudio™ 6 Flex Real-Time PCR System (Applied Biosystems, Foster City, Calif., USA). All reactions were performed on 96-well plates, and a mean value was used to calculate mRNA expression. Results of the experiment are shown in FIGS. 7A and 7B.

As shown in FIGS. 7A and 7B, it is confirmed that when expression of ITGBL1 is increased in chondrocytes, expression of Sox9 and Col2a1 increases, but that when integrin activation is increased by a treatment with Mn²⁺ or DTT, the expression of Sox9 and Col2a1 decreases.

Thus, it is demonstrated that the integrin activation inhibition function of ITGBL1 promotes expression of a chondrogenic gene.

Also, to analyze an influence of the integrin inactivation function of ITGBL1 on the chondrogenesis, a micromass culture experiment was conducted in the following manner. Chondrocytes (mouse chondrocytes; Sigma, 402-05f) were cultured in a DMEM/F-12 medium (gibco, 10565-018, containing 10% (v/v) FBS and 1% (v/v) antibiotics), and were transfected with ITGBL1 DNA using a jetPRIME (Polyplus, 114-15) based on a method of a protocol provided by a manufacturer. A cell mass was formed by the transfected chondrocytes using a micromass method, and was treated with Mn²⁺ or DTT that activate integrins, and with a chondrogenic inducer (TGF-β, dexamethasone, ascorbate-2-phosphate) together with the culture solution, to induce a formation of cartilage tissues. The formation of the cartilage tissues was induced for 7 days, and the cartilage tissues were treated with 4% (w/v) paraformaldehyde and fixed. The fixed cartilage tissues were added to an OCT solution (Cell Path, KMA-0.00-00A) and were sectioned at a thickness of 15 μm using a cryotome (Bright, OTF5000), and an Alcian blue staining was performed, to measure an amount of glycosaminoglycan (GAG). Results of the experiment are shown in FIGS. 7C through 7E.

As shown in FIGS. 7C through 7E, it is confirmed that a size of cartilage increased by overexpression of ITGBL1 is reduced due to an addition of Mn²⁺ or DTT, and accordingly an amount of GAG and a size of cartilage are reduced.

Thus, it is demonstrated that the integrin inactivation function of ITGBL1 promotes the formation of the cartilage tissues.

7-2. Analysis of Expression of Chondrogenic Gene in Case of Increasing ITGBL1 Expression While Inhibiting Expression of Alpha and Beta Subunits of Integrins

To confirm a correlation between integrins and ITGBL1 during chondrogenesis, the following experiment was conducted. Chondrocytes were cultured in an α-MEM (Welgene, LM008-01, containing 10% (v/v) FBS and 1% (v/v) antibiotics), and 2×10⁵ cells were seeded on a 60-mm plate and were transfected with integrin 13-1 siRNA, integrin α-1 siRNA, integrin α-3 siRNA, integrin α-5 siRNA and integrin α-10 siRNA using a jetPRIME (Polyplus, 114-15) based on a method of a protocol provided by a manufacturer, as shown in tables of FIGS. 7F and 7G, to inhibit expression of the above integrins. Also, to express ITGBL1 and inhibit expression of integrins at the same time, the expression of the integrins was inhibited and ITGBL1 DNA was transfected, and a culture was performed at 37° C. RNA extracted from the cells using a PureLink RNA Mini Kit (Invitrogen, 12183018A), and cDNA was synthesized using GoScript Reverse Transcriptase (Promega, A5004A). RT-PCR (BIO RAD, T100 Thermal Cycler) was performed on the synthesized cDNA using Taq polymerase (Coregen, CE-500U). Also, qRT-PCR was performed using a QuantStudio™ 6 Flex Real-Time PCR System (Applied Biosystems, Foster City, Calif., USA). All reactions were performed on 96-well plates, and a mean value was used to calculate mRNA expression.

As shown in FIGS. 7F and 7G, it is confirmed that when expression of alpha and beta subunits of integrins is inhibited, expression of Sox9 increases, and that when the expression of the alpha and beta subunits of the integrins is reduced in a state in which expression of ITGBL1 is increased, the expression of Sox9 further increases.

Based on the results of the experiments conducted in Examples 6 and 7, it is found that the ITGBL1 protein inhibits integrin activation and that the integrin inactivation function of ITGBL1 promotes expression of chondrogenic factors of chondrocytes and a formation of cartilage tissues.

### EXAMPLE 8

### Analysis of Correlation among Integrin Inactivation Function of ITGBL1, Inflammatory Response and Cartilage Degeneration Inhibitory Effect

8-1. Analysis of Expression of Cartilage Degeneration Factor in Case of Increasing ITGBL1 Expression and Activating Integrins

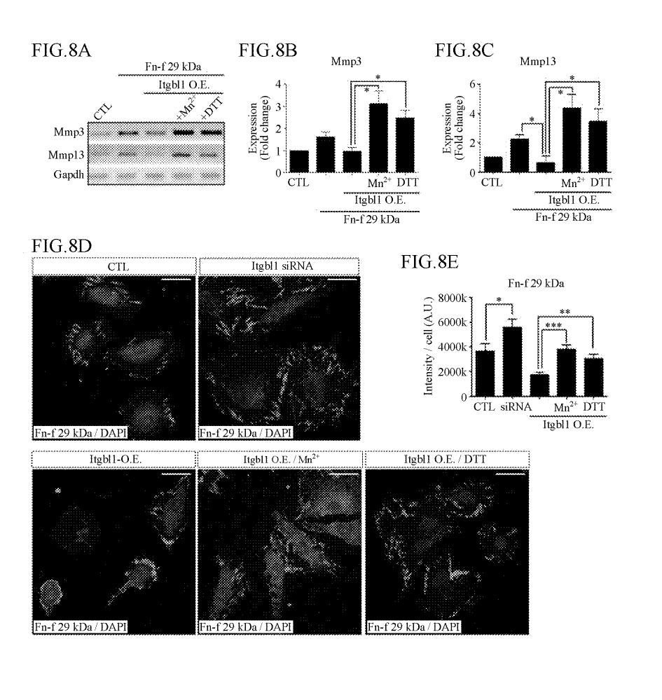

Chondrocytes were cultured in an α-MEM (Welgene, LM008-01, containing 10% (v/v) FBS and 1% (v/v) antibiotics), and 2×10⁵ cells were seeded on a 60-mm plate and were treated with 29-kDa Fn-fs that are fragmented fibronectin known to promote cartilage destruction. The cells were transfected with an ITGBL1 vector using a jetPRIME (Polyplus, 114-15) based on a method of a protocol provided by a manufacturer, were treated with Mn²⁺ or DTT used to activate integrins, and were incubated at 37° C. RNA was extracted from the cells using a PureLink RNA Mini Kit (Invitrogen, 12183018A), and cDNA was synthesized using GoScript Reverse Transcriptase (Promega, A5004A). RT-PCR (BIO RAD, T100 Thermal Cycler) was performed on the synthesized cDNA using Taq polymerase (Coregen, CE-500U). Also, qRT-PCR was performed using a QuantStudio™ 6 Flex Real-Time PCR System (Applied Biosystems, Foster City, Calif., USA). All reactions were performed on 96-well plates, and a mean value was used to calculate mRNA expression. Results of the experiment are shown in FIGS. 8A through 8C.

As shown in FIGS. 8A through 8C, increased expressions of MMP3 and MMP13 by the 29-kDa Fn-fs treatment were reduced when expression of ITGBL1 is increased. Also, when integrins are activated by a treatment with Mn²⁺ or DTT again, expression of MMP3 and MMP13 increases. Thus, it is found that an integrin inactivation function of ITGBL1 reduces expression of MMP3 and MMP13 which are known to cause cartilage destruction and inflammation.

Also, to determine whether an ITGBL1 protein inhibits a binding between chondrocytes and 29-kDa Fn-fs that promote cartilage destruction, the following experiment was conducted. 2×10⁵ cells were seeded on a 60-mm plate and were transfected with ITGBL1 siRNA or DNA using a jetPRIME (Polyplus, 114-15) based on a method of a protocol provided by a manufacturer. 29-kDa Fn-fs (sigma, F9911) were conjugated to the Alexa-488 (Thermo Fisher, A10235) according to the manufacturer's protocol. The Alexa-488 conjugated 29-kDa Fn-fs were treated to the transfected chondrocytes, and the chondrocytes were fixed in 4% paraformaldehyde. The fixed cells were observed using a confocal microscope (Zeiss, LSM880). Results of the experiment are shown in FIGS. 8D and 8E.

As shown in FIGS. 8D and 8E, when expression of ITGBL1 is increased, the binding between the chondrocytes and the 29-kDa Fn-fs is significantly reduced. When treatment with Mn²⁺ or DTT is performed, the binding is increased again. Thus, ITGBL1 may inhibit integrin activation and the binding between the chondrocytes and the 29-kDa Fn-fs, and suppress expression of a cartilage degeneration factor.

8-2. Analysis of Change in Cartilage Degeneration Factor in case of Inhibiting ITGBL1 Expression and Integrin Activation

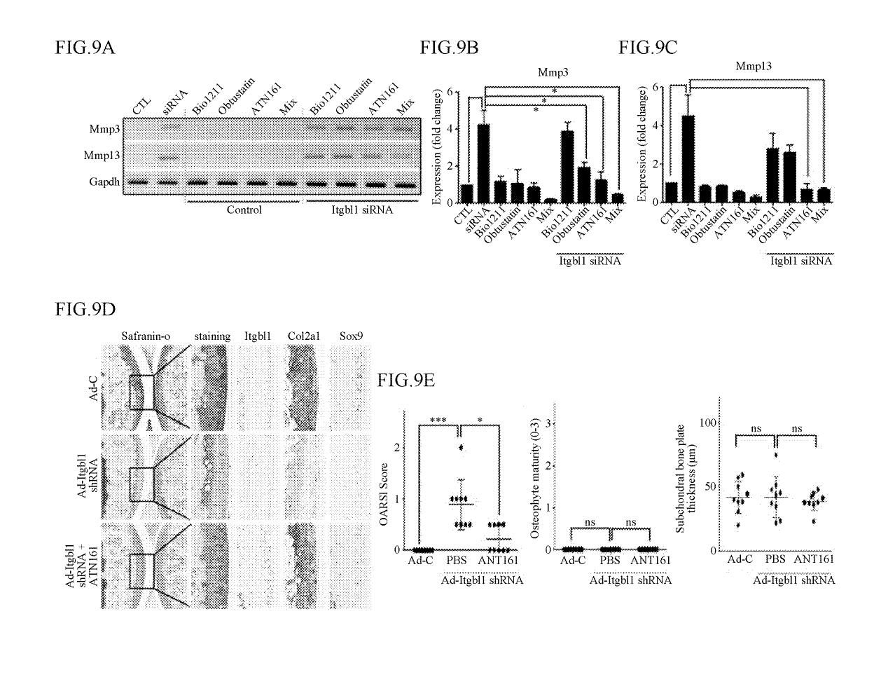

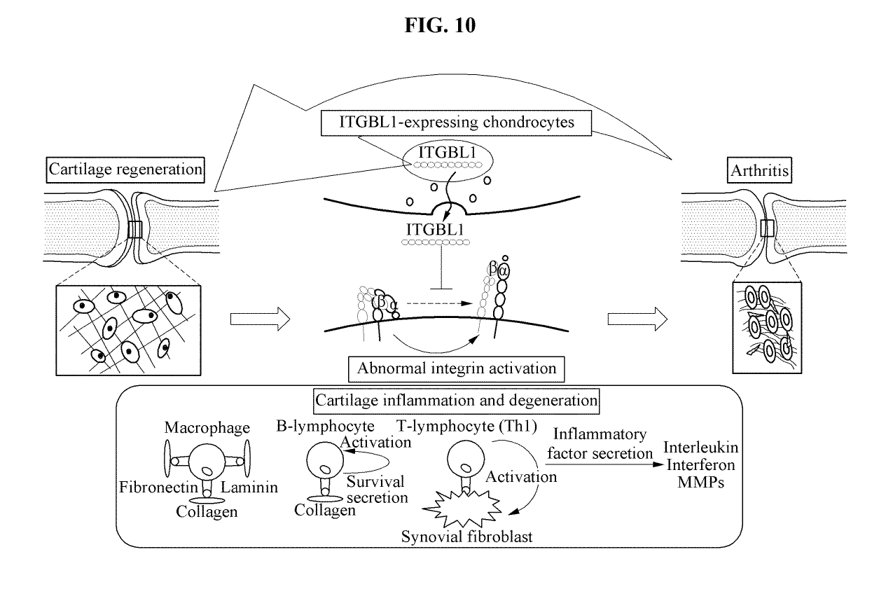

To confirm a correlation between an integrin inactivation function of an ITGBL1 protein and expression of a cartilage degeneration factor, the following experiment was conducted. Chondrocytes were cultured in an α-MEM (Welgene, LM008-01, containing 10% (v/v) FBS and 1% (v/v) antibiotics), and 2×10⁵ cells were seeded on a 60-cm plate and were transfected with ITGBL1-siRNA using a jetPRIME (Polyplus, 114-15) according to the manufacturer's protocol. Bio1211 (TOCRIS, 3910, integrin-α4β1 inhibitor), obtustatin (TOCRIS, 4664, integrin-α1β1 inhibitor), or ATN-161 (TOCRIS, 6058, integrin-α5β1 inhibitor) were added to the α-MEM to inhibit the activation of integrin subtypes, and incubated at 37° C. Total RNAs were extracted from the cells using a PureLink RNA Mini Kit (Invitrogen, 12183018A), and cDNAs were synthesized using GoScript Reverse Transcriptase (Promega, A5004A). RT-PCR (BIO RAD, T100 Thermal Cycler) was performed on the synthesized cDNA using Taq polymerase (Coregen, CE-500U). Also, qRT-PCR was performed using a QuantStudio™ 6 Flex Real-Time PCR System (Applied Biosystems, Foster City, Calif., USA). All reactions were performed on 96-well plates, and a mean value was used to calculate mRNA expression. Results of the experiment are shown in FIGS. 9A through 9C.

As shown in FIGS. 9A through 9C, it is confirmed that when expression of ITGBL1 is inhibited, expression of MMP3 and MMP13 is increased, that when the cells are treated with various types of integrin inhibitors together, the expression of MMP3 and MMP13 is inhibited again, and that ATN-161 among the integrin inhibitors is most effective.

Also, inflammatory responses and cartilage degeneration of ITGBL1 depleted knee joints were analyzed by conducting the following experiment.

Expression of ITGBL1 was inhibited by injecting an ITGBL1-shRNA-containing adenovirus vector (Ad-ITGBL1 shRNA) into a knee joint cavity. Then, ATN-161, an integrin-α5β1 inhibitor, was injected into the knee joint cavity in which expression of ITGBL1 was inhibited by injecting the ITGBL1-shRNA-containing adenovirus vector (Ad-ITGBL1 shRNA), knee joint tissues were excised, and a Safranin 0 staining and immunohistochemistry were performed, so that expression of Col2a1 and Sox9 was observed.

As a control group, an experiment was conducted in the same manner as described above, except that an adenovirus vector (Ad-C) that does not include ITGBL1-shRNA is transduced. Results of experiment are shown in FIGS. 9D and 9E.

As shown in FIGS. 9D and 9E, it is confirmed that depletion of ITGBL1 by intra-articular injection of ITGBL1-shRNA-containing adenovirus vector caused OA-like cartilage degeneration in mouse. Also, it is confirmed that when ATN-161, an integrin-α5β1 inhibitor, is injected into an ITGBL1-depleted mouse knee-joint, the knee cartilage degeneration is recovered.

According to example embodiments, a pharmaceutical composition for preventing or treating cartilage diseases includes, as an active ingredient, at least one of an ITGBL1 protein, ITGBL1 DNA or RNA encoding the ITGBL1 protein. An inhibitor of integrin activation, and a pharmaceutical preparation that includes the pharmaceutical composition as an active ingredient are provided. The pharmaceutical composition that includes at least one of the ITGBL1 protein, ITGBL1 DNA or RNA encoding the ITGBL1 protein as an active ingredient may have an effect of promoting chondrogenesis and inhibiting an inflammation based on a function of an ITGBL1 protein to inhibit integrin activation, and thus it is possible to use the pharmaceutical composition as an effective therapeutic agent for cartilage diseases.

A number of example embodiments have been described above. Nevertheless, it should be understood that various modifications may be made to these example embodiments. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A pharmaceutical composition for preventing or treating cartilage diseases, the pharmaceutical composition comprising:
at least one selected from the group consisting of an integrin beta-like 1 (ITGBL1) protein, ITGBL1 DNA or RNA encoding the ITGBL1 protein, a recombinant vector comprising the ITGBL1 DNA sequences, and recombinant cells transformed with the recombinant vector.

2. The pharmaceutical composition of claim 1, wherein the ITGBL1 protein has an amino acid sequence of SEQ ID NO: 25.

3. The pharmaceutical composition of claim 1, wherein the ITGBL1 has a nucleotide sequence of SEQ ID NO: 26.

4. The pharmaceutical composition of claim 1, wherein the cartilage diseases comprise degenerative arthritis, posttraumatic arthritis, or osteochondritis dissecans.

5. The pharmaceutical composition of claim 1, wherein the recombinant vector is a viral vector or a nonviral vector.

6. The pharmaceutical composition of claim 5, wherein the viral vector is selected from the group consisting of an adenovirus vector, an adeno-associated virus vector, a helper-dependent adenovirus vector, and a retroviral vector.

7. The pharmaceutical composition of claim 1, wherein the recombinant cells are mammalian cells.

8. The pharmaceutical composition of claim 1, wherein the ITGBL1 or the ITGBL1 protein inhibits an integrin-extracellular matrix (ECM) interaction.

9. The pharmaceutical composition of claim 1, wherein the pharmaceutical composition inhibits an inflammation of a cartilage disease.

10. The pharmaceutical composition of claim 1, wherein the pharmaceutical composition promotes chondrogenesis.

11. A pharmaceutical preparation comprising the pharmaceutical composition of claim 1 as an active ingredient.

12. The pharmaceutical preparation of claim 11, further comprising:
at least one selected from the group consisting of a carrier, an excipient and a diluent.

13. The pharmaceutical preparation of claim 11, wherein the pharmaceutical formulation is in a formulation selected from the group consisting of tablets, pills, powders, sachets, elixirs, suspensions, emulsions, solutions, syrups, aerosols, soft or hard gelatin capsules, sterile injectable solutions, and sterile packaged powders.

14. An inhibitor of integrin activation comprising, as an active ingredient, at least one selected from the group consisting of an integrin beta-like 1 (ITGBL1) protein, ITGBL1 DNA or RNA encoding the ITGBL1 protein, a recombinant vector comprising the ITGBL1 DNA sequences, and recombinant cells transformed with the recombinant vector.

15. A method of preventing or treating cartilage diseases, the method comprising:
administering a pharmaceutically effective amount of the pharmaceutical composition of claim 1 to animals other than humans.

16. The method of claim 15, wherein the cartilage diseases comprise degenerative arthritis, posttraumatic arthritis, or osteochondritis dissecans.


SEQUENCE LISTING (26 sequences)

SEQ ID NO: 1 - DNA, 17 bp, Artificial Sequence
  ITGBL1 5′
augcacgcuggagccuu

SEQ ID NO: 2 - DNA, 21 bp, Artificial Sequence
  ITGBL1 3′
aggauauucgcuuccaagcca

SEQ ID NO: 3 - DNA, 25 bp, Artificial Sequence
  ITGBL1 MO
agtagggaagatatacagacctgca

SEQ ID NO: 4 - DNA, 21 bp, Xenopus laevis
uaucggucauagacagcucuu

SEQ ID NO: 5 - RNA, 21 nt, Artificial Sequence
  sence strand of ITGBL1-siRNA
gagcugucuaugaccgauauu

SEQ ID NO: 6 - RNA, 21 nt, Artificial Sequence
  antisence strand of ITGBL1-siRNA
uaucggucauagacagcucuu

SEQ ID NO: 7 - DNA, 1485 bp, Mus musculus
atgcatcctccaggcttcaggaacttcttgttgctggtgtcctcccttctcttcattgggctgtcagctgctcctcaaagcttcttaccatctctgagaagcctgtcgggcgccccctgcaggctgtcccgggcagagtccgaacgcagatgtcgtgcacctgggcagcccccagggagcgctctgtgccatgaccgtggccggtgcgagtgtggggtctgcatctgtcacgtgaccgaacctggcacctacttcggtccactgtgtgagtgccatgagtggatatgcgagacctacgacgggaaaacctgtgcaggccacggtaattgtgactgcggcaagtgcaagtgtgatgtgggatggtctggggaagcttgtcagtacccaaccaagtgtgacctgaccaaaaaaatcagcaaccagatgtgcaagaactcccaagatgtcatctgctccaatgcaggtacatgtcactgtggcaggtgtaagtgtgataattcagatggacatggactcatttatggtaaattttgtgaatgtgatgatagagaatgcatagatgatgaaacagaagaagtatgtggaggccatgggaagtgttactgtggaaactgttactgtgaggctggttggcatggcgataaatgcgagttccagtgtgacatcaccccatgggaaagcaagcgaagatgcacatctccagatggcaaagtctgtagcaacagaggaacatgtgtatgtggtgaatgttcttgccatgatgttgatccaactggggactggggagacattcatggggacacgtgtgagtgtgatgaaagggactgcagagctgtttatgatcgatactctgatgatttctgttcaggtcatgggcagtgtaactgtggaagatgtgactgcagagcaggctggtatgggaagaaatgtgagcacccaaggaattgcccattgtcagctgaggagagcaccaagaagtgccagggtagttctgatcttccttgctctggaaggggcagatgcgaatgtggcagatgcacttgttaccctcctggggacagcagagtctatggcaagacctgtgagtgtgatgaccggcgctgcgaggacctggatggtgtggtctgcggaggccatggcatgtgctcctgtggtcgctgtgtttgtgagaaaggatggtttggtaagctctgccaacacctgcggaagtgtaatatgacagaagaacaaagcaggagtctgtgtgagtcagcagatggcacattgtgctcagggaagggttcttgtcattgtggaaagtgcatttgttctggagaagagtggtatatttcaggggagttttgtgactgtgatgacagagactgtgacaaacacgatggtctcatttgcacagggaatggaatctgtagctgtggaaactgtgaatgctgggatggatggaatggaaatgcatgtgaaatctggcttggtaccgaatatccttaa

SEQ ID NO: 8 - protein, 494 aa, Mus musculus
MHPPGFRNFLLLVSSLLFIGLSAAPQSFLPSLRSLSGAPCRLSRAESERRCRAPGQPPGSALCHDRGRCECGVCICHVTEPGTYFGPLCECHEWICETYDGKTCAGHGNCDCGKCKCDVGWSGEACQYPTKCDLTKKISNQMCKNSQDVICSNAGTCHCGRCKCDNSDGHGLIYGKFCECDDRECIDDETEEVCGGHGKCYCGNCYCEAGWHGDKCEFQCDITPWESKRRCTSPDGKVCSNRGTCVCGECSCHDVDPTGDWGDIHGDTCECDERDCRAVYDRYSDDFCSGHGQCNCGRCDCRAGWYGKKCEHPRNCPLSAEESTKKCQGSSDLPCSGRGRCECGRCTCYPPGDSRVYGKTCECDDRRCEDLDGVVCGGHGMCSCGRCVCEKGWFGKLCQHLRKCNMTEEQSRSLCESADGTLCSGKGSCHCGKCICSGEEWYISGEFCDCDDRDCDKHDGLICTGNGICSCGNCECWDGWNGNACEIWLGTEYP

SEQ ID NO: 9 - DNA, 24 bp, Artificial Sequence
  SOX9-F
aaggagagcgaggaggacaagttc

SEQ ID NO: 10 - DNA, 20 bp, Artificial Sequence
  SOX9-B
tgttcttgctggagccgttg

SEQ ID NO: 11 - DNA, 20 bp, Artificial Sequence
  MMMP3-F
gatgcgcaagcccaggtgtg

SEQ ID NO: 12 - DNA, 23 bp, Artificial Sequence
  MMP3-B
gccaatttcatgagcagcaacga

SEQ ID NO: 13 - DNA, 22 bp, Artificial Sequence
  MMP13-F
aggagcatggcgacttctaccc

SEQ ID NO: 14 - DNA, 24 bp, Artificial Sequence
  MMP13-B
tttgtctggcgtttttggatgttt

SEQ ID NO: 15 - DNA, 19 bp, Artificial Sequence
  Col2al-F
cagttgggagtaatgcaag

SEQ ID NO: 16 - DNA, 20 bp, Artificial Sequence
  Col2al-B
gccttgagcagttcaccttc

SEQ ID NO: 17 - DNA, 21 bp, Artificial Sequence
  MMP-3-F
tcctgatgttggtggcttcag

SEQ ID NO: 18 - DNA, 21 bp, Artificial Sequence
  MMP-3-B
tgtcttggcaaatccggtgta

SEQ ID NO: 19 - DNA, 17 bp, Artificial Sequence
  MMP-13-F
accacatcgaacttcga

SEQ ID NO: 20 - DNA, 17 bp, Artificial Sequence
  MMP-13-B
cgaccatacagatactg

SEQ ID NO: 21 - DNA, 22 bp, Artificial Sequence
  Col2al-F
cacactggtaagtggggcaaga

SEQ ID NO: 22 - DNA, 23 bp, Artificial Sequence
  Col2al-B
ggattgtgttgtttcagggttcg

SEQ ID NO: 23 - DNA, 20 bp, Artificial Sequence
  SOX9-F
catcagcagcaccgcaccca

SEQ ID NO: 24 - DNA, 20 bp, Artificial Sequence
  SOX9-B
cgggtgatgggcgggtagga

SEQ ID NO: 25 - protein, 353 aa, Homo sapiens
MCKNSQDIICSNAGTCHCGRCKCDNSDGSGLVYGKFCECDDRECIDDETEEICGGHGKCYCGNCYCKAGWHGDKCEFQCDITPWESKRRCTSPDGKICSNRGTCVCGECTCHDVDPTGDWGDIHGDTCECDERDCRAVYDRYSDDFCSGHGQCNCGRCDCKAGWYGKKCEHPQSCTLSAEESIRKCQGSSDLPCSGRGKCECGKCTCYPPGDRRVYGKTCECDDRRCEDLDGVVCGGHGTCSCGRCVCERGWFGKLCQHPRKCNMTEEQSKNLCESADGILCSGKGSCHCGKCICSAEEWYISGEFCDCDDRDCDKHDGLICTGNGICSCGNCECWDGWNGNACEIWLGSEYP

SEQ ID NO: 26 - DNA, 4901 bp, Homo sapiens
actctccactgaggggtttggcaccagcaccccgcccagagcagtgccgctgcccaaatcctcgcaggcagctcatcaacgcaattgcaactccggctggagccccggacctgcaagcctgggtgtccgtgggtccgtctgcccagccatctgctggtggcacctctccctcctgccgcctccctcggtgaaccccaccttgcagaagtgcagctcgcccggagcagcccaggagctcagcatgcgtcccccaggcttcaggaacttcttgctgctggcgtcctcccttctctttgctgggttgtcagctgttcctcaaagcttctcgccatctctgaggccatggtaagtgtgactgtggcaagtgcaagtgtgaccagggatggtatggggatgcttgccagtacccaactaactgtgacttgacaaagaagaaaagtaaccaaatgtgcaagaattcacaagacatcatctgctctaatgcaggtacatgtcactgtggcaggtgtaagtgtgataattcagatggaagtggacttgtgtatggtaaattttgtgagtgtgacgatagagaatgcatagacgatgaaacagaagaaatatgtggaggccatgggaagtgttactgtggaaactgctactgcaaggctggttggcatggagataaatgtgaattccagtgcgatatcaccccctgggaaagcaagcgaagatgcacgtctccagatggcaaaatctgcagtaacagagggacttgtgtatgtggtgaatgtacctgtcacgatgttgatccgactggggactggggagatattcatggggacacctgtgaatgtgatgagagggactgtagagctgtctatgaccgatattctgatgacttctgttcaggtcatggacagtgtaattgcggaagatgtgactgcaaagcaggctggtatgggaagaagtgtgagcacccacagtcctgcacgctgtcagctgaggagagcatcaggaagtgccagggaagctcggatctgccttgctctgggaggggtaaatgtgaatgtggcaaatgcacctgctatcctccaggagatcgccgggtgtatggcaagacttgtgagtgtgatgatcgccgctgtgaagacctcgatggtgtggtctgtggaggccacggcacatgttcctgtggtcgctgtgtttgtgagagaggatggtttggaaagctctgccaacatccgcggaagtgtaacatgacggaagaacaaagcaagaatctgtgtgaatcagcagatggcatattgtgctcggggaagggttcttgtcattgtgggaagtgcatttgttctgctgaagagtggtatatttctggggagttctgtgactgtgatgacagagactgcgacaaacatgatggtctcatttgtacagggaatggaatatgtagctgtggaaactgtgaatgctgggatggatggaatggaaatgcatgtgaaatctggcttggctcagaatatccttaacaattacatgagagagcaattttgatgacgtattcgctgatgcacagacattggaattcattacattatctagccatcgtaatacaaatgacctttggatttctctatcacggcgcttatcctcctattaaggatcagaaagttaaactttacggaccacgtacgtccctgtctcatattcttctttgttggtttctgtttaatactttaaagctgtaaaaaaaatttaaaaaattccttctgagctcccaggccatacaaaagcaacttaagggtggatttggctggcagctgggcagtactttgtcagcctcagctccactggaactgcccttgattctctatagctcatcacaaaacctcaggtatgtcttatttttcttataatcctctgtatgttattatcctttttattaacaacaacaactacaacaaaagtgctgctctttttcatagtctcatgtaagaagagtgtcaaaaatgtcctcggcatttgttttctcagaaaaagaatttagatattggagaaattatcataccataaaaagttcaaaatgtgattcatctaaaattttggacaaagatgaaaatatagtcagctctgttctagtgttcatttagttagaaaccatagtaccggaactgtattatagtagaagttaatggtacctaaactctaaggaatgctttttctttgaaaacagaaatatttttacttatttctgtccactatgtttgtgtgtatgtgtgtgtttgtgtgtgtatgtgtggttttgatgccagcaatgcaaacaccaatatatctattaagaagtgattgaatttagattggaaaaaataaaatagttacatgtaaagacataaactctttccttcttaaatgtgattttcaactctatgatagaaactccctctaaaaaacaagcagggcaagaacaaacttacttgtaatgggtgagagaaaataagcaaatctcattggctcattcctgtttttaggaagttgcagtgggattaatgtcaattggaagggatgcagcaagtttagagaaggtgttggcactaacgctgcttgtttggcaaatcatcatcactgaggtattccacccagagactttttcaaaaaagtcaactaaagtgctaagtcataagaggagagccattatacccgttgtctttctgggctccttgagtttatctggattccaacagcacttggaaagtaccgccctccactacctcaaatgcaaacacaatctctgccagtagacattggaattagacttagtgaaaaccaggaaaaaaaaaaaaaactaaaatataactcagccttagtttgcagactcaactgtcaacactttgaaaccctactccatattcaagagagtcaacaggccctatgtcacattgtaggctaagaaacctcagctatgactactatttcactcaaagtaagaccatgtacatcctggattttgatatgcctgcattttggaaaaaaaaattgaggatttagcccagtcgctttcccttgatatagccttgttctgaattaaaatggaaaaaatatttcaattattttcatcttgttctcaactggatgttagtcaatgtggtctctttttgaatatgtatcaaatattaatgatccataatattagggagataattttaaatgaagtgatacgtgtctaacttacttaaagaataagtttttggtttttgcttttaaagacaaccaaatctgatattgttcatcctgataaaaataacaacttttagtgcttaaagcattaattaagcaagtggctaggtatgataaagaacttctgcttgctccccaagaggcaaactattagaagaactggagcgggagtcctttggacccatcgtggatctctttaagccactgctacccaaaaacattcaggacaagcaaacatttagagcaagaatctccaaattcttcaggatttgtaatgaaatgatgttcagttccattttgctctttacatagggtggagaattgtcatgtcttctctaatttttccaagtaaagtggtagcaaaatgttttaaaaagcaatcttatattagaaaacaaaaatgttgtcacttgaaataccaaaacaacatttctgagcgttgttgagggactggcaaagcaatcagctactataacaaatcagtagaaataaccctcccacaccagatatgcatgcagaaggaatggagtattatagagacttgatacaatggacatatgcacatggaggtacaaaacacacagtctaaatacaaatgaattccatcagatttactatacggaacatcagtagtgacagattgcacttcttacttaataacagcaaacttaatttctgaggggaaaaaaatggcgaagtcttatcccaaacaaatagcaagagaggtatcatcaaagagctaaaattttctttggcatggtaaagggggaaattgagtttaccaacttatttacatgacatttctctatattggtgagtaatgcaatgccattttgttacataaagttgtttgatgttttttaatatgccttcatataaatattttattcaatatgttgtatttgtgaatttaacaaatgatattaaacacaaactacaatgcagacagacaaactctttgtatgcaaattagcaatacataccaacagttcttgatacacaggtacctactacatgcagctcatcattgctgtcctcttcccatgctacaggtgagaccagacacaaagtaaatgacctaactcaattacaaataacaaggacccctctgcaatatgtctaaacatatattagaagaaagtatttgacatccttcatagggataaatgcccttatagacaccccatatataaaacacaacagagatacacatttacataaatctcagtcatttacaaaaataaatcttgtcttaatttaaaccaataaacagacttgcaggggaaaaagaaaccagcaagtagggctaattatcagtaacaaatagatgggggtgtttgctctgtgtgtgtgtgtgtgtgtgtgtgtgtgtgtgtgtatatgtgtgtgtttgtgtgaagtgaagtgttgctgctgtaagtagtgtccataagcccatttgactgtattacaagttagttaattactcatatagttggccacatattatgggatctatgttttctgaaaataattggttaatggaagttatctaatatattttaactgttcctgttaaaaacaataggcttcaagatgacataacaccaaatcaaaaatgaccaaaggaatcattttgtttgttagatttgtaatttagcatcattggcaataaatctactcaaacgttcaaaaaaaaaaaaaaaaaa